(19) United States
(12) Patent Application Publication
(10) Pub. No.: US 2015/0098925 A1
(43) Pub. Date: Apr. 9, 2015

(54) COMPOSITIONS AND METHODS FOR TREATING CARDIOVASCULAR DISEASES USING DISEASE-SPECIFIC PROMOTER

(71) Applicant: Kiromic, LLC, Lubbock, TX (US)

(72) Inventor: Maurizio Chiriva-Internati, Lubbock, TX (US)

(21) Appl. No.: 14/508,642

(22) Filed: Oct. 7, 2014

Related U.S. Application Data
(60) Provisional application No. 61/887,704, filed on Oct. 7, 2013.

(51) Int. Cl. C12N 15/86

(52) U.S. Cl. CPC C12N 15/86; C12N 2710/10033
USPC 424/85.2; 514/44 R; 514/44 A; 435/320.1

(57) ABSTRACT

The methods and systems of the present invention provide for an expression vector containing a disease-specific promoter linked to a gene encoding a therapeutic agent, such as a protein, microRNA, siRNA or other therapeutical molecule, e.g., other oligonucletide. A variety of different promoters may be used with the present invention, provided that the promoter preferentially expresses the gene linked to it at the site of the disease and not more globally within the body. The disease-specific promoter may be the promoter of the LOX1 gene. The therapeutic agent may be Interleukin 10 (IL10).

### CROSS REFERENCE TO RELATED APPLICATION

This application claims priority to U.S. Provisional Application No. 61/887,704 filed on Oct. 7, 2013, which is incorporated herein by reference in its entirety.

### GOVERNMENT LICENSE RIGHTS

This invention was made with government support under Grant No. R56 AI093695 awarded by the National Institutes of Health and under Merit Review grant awarded by the U.S. Department of Veterans Affairs. The government has certain rights in the invention.

### FIELD OF THE INVENTION

The present invention relates to methods and compositions for the treatment of cardiovascular diseases and other conditions. In particular, the present invention relates to gene therapy using disease-specific promoters in treating cancer.

### BACKGROUND OF THE INVENTION

The hallmark goal of gene therapy is to deliver a therapeutic gene which then acts to counteract a negative phenotype or disease within the patient or animal model. While there are many types of diseases to treat, each disease is somewhat different and there are a variety of delivery/expression strategies which can be undertaken. There are two primary types of gene expression approaches that may be used to drive gene expression, (i) constitutive and (ii) tissue specific. The issue is that many, perhaps all, therapeutic genes will likely have negative consequences when expressed, i.e., adverse reactions, especially if the genes are expressed at high levels. Nevertheless, the gene therapy agents must be safe, and not induce wide-spread unintended damage.

The first major expression approach is the “constitutive” approach, such as using the cytomegalovirus (CMV) immediate early promoter (“pr”). The treatment of genetic syndromes might be the most appropriate for this approach. Genetic syndromes result from a faulty protein which is important within a tissue or organ for normal function. For genetic syndromes the goal of gene therapy is simple; get that therapeutic gene and its protein delivered and expressed into as many cells of the patient, organ or tissue as possible to give back normal function. The strategy then is usually for maximum gene delivery and gene expression.

However, many, if not most, therapeutic genes actually have a physiological down-side because of their inherent function and adverse reactions when expressed at high levels. For example, in the case of inflammatory diseases, such as cardiovascular disease, therapeutic genes that have been used in the treatment of atherosclerosis, in particular, interleukin 10 (IL10, IL-10) (which, while strongly immuno-suppressive), is also associated with a number of adverse reactions which are manifested in the clinical setting and in some animal models. These adverse reactions include increased bacterial, fungal and viral infections, cancer, headache, and anemia (8-16). In general, the strongest therapeutic genes are also likely to be the most dangerous and they must be tightly regulated.

A major refinement of the constitutive approach is the “tissue-specific” approach. Here, the delivered transgene is expressed only within a specific cell type, thereby limiting its overall expression (17). Although this approach represents an improvement over the constitutive approach, it appears to a modified version of the constitutive approach, but, limited to a specific cell type.

The third approach is a “disease-specific” approach (4). The goal of the disease-specific approach is to limit the expression of the therapeutic transgene to the site of disease, in the cells which are changing towards the disease-associated phenotype. This disease-specific approach gives the gene therapy an important safety feature for gene therapy against adverse reactions from the over-expression of a powerful therapeutic transgenes such as IL10.

Because of the potential advantages of the disease-specific approach, there is a continuing need to develop and test new expression systems.

### SUMMARY

The present invention provides for an expression vector for treating a cardiovascular disease, e.g., atherosclerosis. The promoter of the present expression vector is a disease specific promoter.

The present invention also provides for a method of treating a cardiovascular disease in a subject (having the cardiovascular disease, e.g., atherosclerosis), comprising the step of administering to the subject a therapeutically effective amount of an expression vector.

The vector may comprise a promoter comprising (or consisting of) a nucleotide sequence about 80% to about 100% identical to the nucleotide sequence of SEQ ID NO: 1.

The promoter of the vector may comprise (or consist of) the nucleotide sequence of the promoter of the lectin-like oxidized low density lipoprotein receptor 1 (LOX-1) gene. In one embodiment, the promoter comprises the nucleotide sequence of SEQ ID NO: 1.

The vector may also comprise a therapeutic gene, under the control of the promoter, encoding a therapeutic agent which is therapeutically effective to treat the cardiovascular disease. The therapeutic agent can be a protein, a microRNA or an siRNA. For example, the therapeutic agent may be Interleukin 10 (IL-10). In one embodiment, the therapeutic agent is Interleukin 10 encoded by a gene comprising the nucleotide sequence of SEQ ID NO: 2.

The expression vector may be an adeno-associated virus (AAV) vector (derived from AAV). For example, the AAV vector may be an AAV2 or AAV8 vector. The AAV vector may comprise an AAV8 capsid gene which can be wild-type or mutated. In one embodiment, the AAV8 capsid gene of the AAV vector comprises SEQ ID NO: 3; SEQ ID NO: 5; or (iii) SEQ ID NO: 7.

### BRIEF DESCRIPTION OF THE FIGURES

FIG. 1: Structure of AAV vectors and experimental scheme. A. is a cartoon showing the basic structure of the three AAV vectors used in this study. B. shows the experimental scheme and data collected.

FIG. 2: More detailed structure of the AAV.LOX1pr-hIL10 vector plasmid. This figure shows in more detail the structure of the basic structure of the AAV2.LOX1pr-hIL10 vector plasmid used to generate the virus. Note that the relative positions of three responsive elements for AngII, Ox-LDL and PMA are shown within the promoter sequence. However, the full length LOX1pr is likely responsive to many other stimuli as well.

FIG. 3: Physical characterization of animal groups. Data shown are mean+/−SE. A. shows the levels of total cholesterol. Note that cholesterol levels of all the animals on HCD had had significantly higher cholesterol levels than ND animals. B. shows the animal weights at the end of the experiment. The key at the bottom is used for both panels.

FIG. 4: Expression of hIL10 in animal groups. Relative expression of delivered hIL10 genes compared to endogenous β-actin determined by real-time quantitative PCR from aorta of 6 mice in each group. For QRT-PCR the quantity of mRNA for each vector transgene was normalized to βactin in the same sample. Data shown are mean+/−SE. Note that while CMVpr-IL10 (CMV promoter-IL10) and LOX1pr-IL10 treatments were statistically similar, however LOX1pr-IL10 trended to be less than half the expression level of CMVpr-IL-10. Disease-specific Expression of hIL10 in various organs: LOX-1 promoter is predominantly expressed in the aorta, the site of disease (57).

FIG. 5: Systolic blood velocity. High resolution ultrasound (HRUS) was used to measure blood flow velocities in the luminal center of the abdominal region of the aorta in 8-10 animals from each group. Shown is a quantification of the results. Note that both CMVpr-hIL10- and LOX1pr-hIL10-HCD-treated animals all had significantly lower blood velocity than the AAV/Neo-HCD-treated animals, similar to ND controls. However, CMVpr-hIL10-HCD- and LOX1pr-hIL10-HCD-treated animals were statistically similar to each other.

FIG. 6: Analysis of the aortic lumen by high resolution ultrasound (HRUS). HRUS was used to measure the cross sectional area of the thoracic region of the aortas in 8-10 animals from each animal group. Shown is a quantification of the cross-sectional area for the abdominal/thoracic region of the aorta. Note that both CMVpr-hIL10- and LOX1pr-hIL10-HCD-treated animals had a significantly larger cross sectional area than the AAV/Neo-HCD-treated animals, indicating significant efficacy. However, CMVpr-hIL10-HCD- and LOX1pr-hIL10-HCD-treated animals were statistically similar to each other.

FIG. 7: Analysis of the aortic wall thickness by HRUS. HRUS was used to measure the wall thickness of the aorta. Shown is a quantification of the thoracic region of the aortas in 8-10 animals from each animal group. Note that both CMVpr-hIL10- and LOX1pr-hIL10-HCD-treated animals had a significantly thinner aortic wall than the AAV/Neo-HCD-treated animals, indicating significant efficacy. However, CMVpr-hIL10-HCD- and LOX1pr-hIL10-HCD-treated animals were statistically similar to each other.

FIG. 8. Transduction of human primary placental umbilical cord vein rings in culture by AAV2 and modified AAV2-EYH. Vein rings were infected with 10⁸ AAV2-CMV-EYH/eGFP virus in vitro. Photomicrographs were taken 2 days post-infection. Note that the smooth muscle cells are strongly targeted by the AAV2-EYH-modified capsid.

FIG. 9. Specific changes in the AAV8 capsid for improved transduction of arterial smooth muscle cells, including tasks 1 and 2. The indicated changes will be made in pDG8). Tyrosines at position 447 and 733 will be replaced by phenylalanines, while the EYH peptide (a peptide having seven amino acids), will be inserted after position 590. These modifications may enhance delivery of the AAV2/8(cvd1). LOX1pr-IL10 DNA into vascular smooth muscle cells.

### DETAILED DESCRIPTION

The methods and systems of the present invention provide for an expression vector containing a disease-specific promoter linked to a gene encoding a therapeutic agent, such as a protein, microRNA, siRNA or other therapeutic molecule, e.g., other oligonucleotide. A variety of different promoters may be used with the present invention, provided that the promoter preferentially expresses the gene linked to it at the site of the disease and not more globally within the body.

Because of the adverse reactions which can arise from the use of certain therapeutic agents, their overall production must be limited. The use of disease-specific transcriptional promoters is one approach to give such reduced and selective expression. Such disease-specific gene expression should direct expression to the site of disease where it is most needed and, at the same time, limit overall production. The disease-specific expression of a therapeutic agent can lower the possibility of significant adverse reactions (17-23).

The present invention provides for an expression vector for treating a variety of conditions including cardiovascular diseases, e.g., atherosclerosis. The expression vector may be an adeno-associated virus (AAV) vector (derived from AAV). In one embodiment, the disease-specific promoter is the promoter of the LOX-1 gene (LOX-1 promoter, LOX1 promoter, or LOX1pr) (17, 4). The therapeutic agent may be Interleukin 10 (IL10).

### Disease-Specific Promoters

The present expression vector contains a disease-specific promoter. A disease-specific promoter can ensure that the expression of a gene under its control is substantially limited to diseased tissues or tissues surrounding diseased tissues. The promoter of any gene, which is up-regulated during a disease, may be used as a disease-specific promoter in the present invention. Under normal conditions (a non-disease state), the gene, under the control of its promoter, is expressed at a low level which may or may not be detectable. During a disease, the promoter is activated by disease-associated stimuli (e.g., blood sheer stress, dislipidemia, immune cell trafficking, other activations, etc.), and the promoter/gene is strongly up-regulated. The use of a disease-specific promoter also is an inherent safeguard against the adverse effects of a therapeutic agent under its control. Once the disease becomes diminished, the disease-stimulus will be eliminated or reduced (e.g., after the gene therapy). In response, the expression of the therapeutic agent, under the control of the disease-specific promoter, should be down-regulated, resulting in a nature negative feedback.

Lectin-like oxidized low density lipoprotein receptor 1 (LOX1, LOX-1, oxidized LDL receptor 1, OLR1) is a scavenger receptor which is expressed when cells become activated, and binds many ligands. LOX1 is also one of the receptors which recognize oxidized low density lipoprotein (Ox-LDL). Moreover, it is expressed in many cell types, including endothelial cells, smooth muscle cells, and macrophages, the major cell types believed involved in atherosclerosis (23-28). It is heavily expressed within atherosclerotic plaque. LOX-1 is also considered one of the earliest markers of activated endothelium, which predates the initiation of atherosclerosis (17, 24). LOX1 appears to be transcriptionally up-regulated during atherogenesis. Many agents, such as Ox-LDL and AngII, induce its up-regulation.

In one embodiment of the present invention, the LOX-1 transcriptional promoter (the promoter of the LOX1 gene, LOX1pr) is used as a disease-specific promoter of the expression vector for expressing therapeutic genes to counter a cardiovascular disease (e.g., to counter atherogenesis). The LOX-1 promoter can be from human or from other species (4, 17).

In another embodiment, the present expression vector contains a promoter comprising/consisting of (or consisting essentially of) a nucleotide sequence about 60% to about 100%, about 70% to about 100%, about 80% to about 100%, about 85% to about 100%, about 90% to about 100%, about 95% to about 100%, about 98% to about 100%, about 60%, about 70%, about 80%, about 85%, about 90%, about 95%, about 98%, or about 100% identical to the nucleotide sequence of SEQ ID NO: 1.

Non-limiting examples of the disease-specific promoters also include the promoters of the gene of Apoliporotein E (apoE), superoxide dismutase 2 (SOD2), carcinoembryonic antigen, HER-2/neu, DF3/MUC (Dachs, et al. 1997. Oncol. Res. 9:313-25), tyrosinase, fetoprotein, albumin, CC10, or prostate-specific antigen; tet-responsive element, or Myc-Max response elements. Promoters that may also be used in the present invention include the promoters disclosed in Eyster et al., Gene expression signatures differ with extent of atherosclerosis in monkey iliac artery, Menopause, 2011, 18(10):1087-95. Wilcox et al., Local expression of inflammatory cytokines in human atherosclerotic plaques, J Atheroscler Thromb. 1994; 1 Suppl 1:S10-3. The disclosures of both are incorporated herein by reference in their entirety.

The promoter may be wild-type or a mutant. Mutants can be created by introducing one or more nucleotide substitutions, additions or deletions. For example, mutations can be introduced by standard techniques, such as site-directed mutagenesis and PCR-mediated mutagenesis.

Under the control of the disease-specific promoter, the expression level of a therapeutic gene (encoding a therapeutic agent) in the tissue involved in the condition being treated may be lower than the expression level of the therapeutic gene under the control of a constitutive promoter. In one embodiment, the expression level of a therapeutic gene (encoding, e.g., IL-10) in the blood vessel (e.g., the aorta, an artery, artery wall, etc.) may be about 90%, about 80%, about 70%, about 60%, about 50%, about 40%, about 30%, about 20%, less than about 90%, less than about 80%, less than about 70%, less than about 60%, less than about 50%, less than about 40%, less than about 30%, or less than about 20%, of the expression level of the therapeutic gene under the control of a constitutive promoter (e.g., the cytomegalovirus (CMV) immediate early promoter).

In another embodiment, the expression level of a therapeutic gene (encoding a therapeutic agent) in the tissue not involved in the condition being treated may be lower than the expression level of the therapeutic gene under the control of a constitutive promoter. For example, the expression level of a therapeutic gene (encoding, e.g., IL-10) in the tissue not involved in the condition being treated (e.g., the lungs or liver) may be about 90%, about 80%, about 70%, about 60%, about 50%, about 40%, about 30%, about 20%, about 10%, about 5%, about 2%, about 1%, less than about 90%, less than about 80%, less than about 70%, less than about 60%, less than about 50%, less than about 40%, less than about 30%, less than about 20%, less than about 10%, less than about 5%, less than about 2%, less than about 1%, of the expression level of the therapeutic gene under the control of a constitutive promoter (e.g., the cytomegalovirus (CMV) immediate early promoter).

The level of a protein may be determined by any suitable assays, including, but not limited to, using antibodies specific to the protein in Western blot, enzyme-linked immunosorbent assay (ELISA), flow cytometry, immunocytochemistry or immunohistochemistry.

Gene expression levels may be assayed by any suitable methods, including, but not limited to, measuring mRNA levels and/or protein levels. Levels of mRNA can be quantitatively measured by RT-PCR, Northern blot, next-generation sequencing, etc.

### Therapeutic Genes

The vectors of the present application may further comprise a heterologous gene under the control of the promoter. The gene may be a therapeutic gene encoding a therapeutic agent. As used herein, the phrase “therapeutic agents” refer to any molecules or compounds that assist in the treatment or prevention of a disease.

The therapeutic agent that may be expressed under the control of the disease-specific promoters can be a protein, a polypeptide, a polynucleotide (e.g., a small interfering RNA (siRNA), a microRNA (miRNA), a ribozyme or an antisense molecule), an antibody or antigen-binding portion thereof, or any other molecules. The therapeutic agent may be cytokines, immunoglobulins (e.g., IgG, IgM, IgA, IgD or IgE), integrins, albumin or any other potentially, therapeutically relevant molecule. The choice of the particular molecule is determined by the pathology of a particular disease. Non-limiting examples of the therapeutic agents include Interleukin 1 (IL-1), IL-2, IL-3, IL-4, IL-5, IL-6, IL-7, IL-8, IL-9, IL-10, IL-11 IL-12, GM-CSF and G-CSF.

In one embodiment, therapeutic agents for treating atherosclerosis include agents which down-regulate the immune system, agents which control dyslipidemia (eg. high cholesterol), and agents which down-regulate anti-reactive oxygen species (anti-ROS). For example, the present therapeutic agents may include transforming growth factor beta 1 (TGFβ1), SMAD3, interleukin 10 (IL-10), STAT3, Netrin-1, angiotensin II type 2 receptor (AT2R), an anti-reactive oxygen species (anti-ROS) agent (e.g., peroxiredoxin 6 (PRDX6) which is an anti-thiol-ROS protein), apolipoprotein A1 Milano (ApoA1 milano) which affects dyslipidemia. In one embodiment, the IL-10 gene used in the present vector consists of/comprises the nucleotide sequence of SEQ ID NO: 2.

Non-limiting examples of therapeutic agents also include a cytokine, a chemokine, an immunomodulatory molecule, a prodrug converting enzyme, an angiogenesis inhibitor, an angiogenesis promoter, a toxin, an antitumor agent, a mitosis inhibitor protein, an antimitotic agent, a transporter protein, tissue factor, enzymes, blood derivatives, hormones, lymphokines, interleukins, interferons, tumor necrosis factors, growth factors, neurotransmitters or their precursors or synthetic enzymes, trophic factors (such as BDNF, CNTF, NGF, IGF, GMF, alpha-FGF, beta-FGF, NT3, NTS, and HARP/pleiotrophin), apolipoproteins (such as ApoAI, ApoAIV, ApoE, dystrophin or a minidystrophin), the CFTR protein associated with cystic fibrosis, intrabodies, tumor-suppressing genes such as p53, Rb, Rap1A, DCC, k-rev, coagulation factors such as factors VII, VIII, IX, DNA repair factors, suicide agents which cause cell death, cytosine deaminase, and pro-apoptic agents.

The therapeutic gene encoding the therapeutic agent may be from human or other species.

The therapeutic gene may be wild-type or a mutant. Mutants can be created by introducing one or more nucleotide substitutions, additions or deletions. For example, mutations can be introduced by standard techniques, such as site-directed mutagenesis and PCR-mediated mutagenesis. When the therapeutic agent is a protein, conservative amino acid substitutions may be made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid, asparagine, glutamine), uncharged polar side chains (e.g., glycine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

For treating atherosclerosis, anti-inflammatory cytokines such as transforming growth factor beta 1 (TGFβ1) or interleukin 10 (IL-10) might be used for therapeutic effect. However, these particular agents have significant adverse reactions (23, 24). For example, the phenotype of TGFβ1 is very pleiomorphic and is highly associated with the induction of fibrosis (26-28). In comparison, I-10 has fewer complications yet is still associated with a number of adverse reactions such as headache, anemia, and increased infections (25-30). Because of the adverse reactions associated with the use of IL-10, its overall production must be limited. The use of disease-specific transcriptional promoters is one approach to give such reduced and selective expression. Such successful disease-specific gene expression should direct expression to the site of disease where it is most needed and, at the same time, limit overall production. The disease-specific expression of IL-10 can be contemplated to lower the possibility of significant adverse reactions as has been reported to occur (22, 27).

Variants, analogs, or fusion proteins of a therapeutic agent may be used. In one embodiment, the therapeutic agent comprises or consists of the nucleotide sequence of SEQ ID NO: 2. In another embodiment, the therapeutic agent comprises or consists of a nucleotide sequence about 60% to about 100%, about 70% to about 100%, about 80% to about 100%, about 85% to about 100%, about 90% to about 100%, about 95% to about 100%, about 98% to about 100%, about 60%, about 70%, about 80%, about 85%, about 90%, about 95%, about 98%, or about 100% identical, to the nucleotide sequence of SEQ ID NO:2.

The therapeutic agent may be a small interfering RNA (siRNAs) or small-hairpin RNA (shRNA). The siRNA or shRNA may reduce or inhibit expression of a therapeutic target. SiRNAs may have 16-30 nucleotides, e.g., 16, 17, 18, 19, 20, 21, 22, 23, 24, 25, 26, 27, 28, 29, or 30 nucleotides. The siRNAs may have fewer than 16 or more than 30 nucleotides.

In another embodiment, the therapeutic agent is an antisense polynucleotide. The antisense polynucleotide may bind to a therapeutic target. An antisense oligonucleotide can be, for example, about 7, 10, 15, 20, 25, 30, 35, 40, 45, 50, 55, 60, 65, 70, 75, 80, or more nucleotides in length.

In a further embodiment, the therapeutic agent is a ribozyme. U.S. Pat. Nos. 8,592,368 and 5,093,246. Haselhoff et al., Nature 334: 585-591 (1988).

The therapeutic agent may be an antibody or antigen-binding portion thereof that is specific to a therapeutic target. The antibody or antigen-binding portion thereof may be the following: (a) a whole immunoglobulin molecule; (b) an scFv; (c) a Fab fragment; and (d) an F(ab′)2 etc. The antibody or antigen-binding portion thereof may be monoclonal, polyclonal, chimeric and humanized.

### Vectors

As used herein, the term “vector” refers to a polynucleotide capable of transporting another nucleic acid to which it has been linked. The present vectors can be, for example, a plasmid vector, a single- or double-stranded phage vector, or a single- or double-stranded RNA or DNA viral vector. Such vectors include, but are not limited to, chromosomal, episomal and virus-derived vectors, e.g., vectors derived from bacterial plasmids, bacteriophages, yeast episomes, yeast chromosomal elements, and viruses such as baculoviruses, papova viruses, SV40, vaccinia viruses, adenoviruses, fowl pox viruses, pseudorabies viruses and retroviruses, and vectors derived from combinations thereof, such as those derived from plasmid and bacteriophage genetic elements, cosmids and phagemids.

Expression vectors can be used to replicate and/or express the nucleotide sequence encoding a therapeutic agent in a target mammalian cell. A variety of expression vectors useful for introducing into cells the polynucleotides of the inventions are well known in the art. Viral vectors include, but are not limited to, adeno-associated virus, adenovirus, vaccinia virus, alphavirus, retrovirus and herpesvirus vectors.

In one embodiment, the vector is an adeno-viral associated virus (AAV) vector. Without wishing to be bound by any specific theory or mechanism, it is believed that the promoter can function in a disease-specific manner within the AAV vector due to the lack of strong promoter elements within the AAV inverted terminal repeats (35, 36). See also, e.g., Walsh et al., 1993, Proc. Soc. Exp. Biol. Med. 204:289-300; U.S. Pat. No. 5,436,146). AAV has been known to be an effective vector since 1984 (31, 32, 33-38), including delivering the IL10 gene in mouse atherogenesis models (26, 27).

Any of the AAV serotypes may be used, including, but not limited to, AAV1, AAV2, AAV3, AAV4, AAV5, AAV6, AAV7, AAV8, AAV9, AAV10, AAV11, etc.

The present vector may comprise wild-type or mutant AAV capsid (e.g., AAV2, AAV8, etc.) encoded by the AAV cap open reading frame (ORF).

In one embodiment, the mutant AAV capsid may have at least one tyrosine residue mutated (e.g., deleted, substituted with other amino acid residue(s), or having amino acid residue(s) inserted adjacent to the tyrosine). In another embodiment, there is an insertion of at least one amino acid residues into the cap ORF or the capsid gene. The mutant AAV capsid may have a combination of two or more of the above mutations.

For example, the AAV2 capsid may have Tyrosine-444 replaced by phenylalanine, and/or Tyrosine-730 replaced by phenylalanine. The AAV8 capsid may have Tyrosine-447 replaced by phenylalanine, and/or Tyrosine-733 replaced by phenylalanine SEQ ID NO: 4 shows the amino acid sequence of AAV8 capsid with both tyrosine residues at positions 447 and 733 being substituted with phenylalanine residues. SEQ ID NO: 3 shows the nucleotide sequence of AAV8 capsid with both tyrosine residues at positions 447 and 733 being substituted with phenylalanine residues.

In another example, a 7-amino acid peptide having the sequence EYHHYNK (SEQ ID NO: 13) (refereed to as “EYH” peptide herein), is inserted into the AAV capsid. The AAV2 capsid may have the peptide having the sequence EYHHYNK inserted after amino acid position 588. The AAV8 capsid may have the peptide having the sequence EYHHYNK inserted after amino acid position 590 (e.g., see SEQ ID NO: 5 for the nucleotide sequence of this embodiment; and SEQ ID NO: 6 for its amino acid sequence).

In a third embodiment, the AAV capsid may have a combination of two or more of the above modifications. SEQ ID NO: 7 shows the modified AAV8 capsid nucleotide sequence (SEQ ID NO: 8 shows the modified AAV8 capsid amino acid sequence) having both (1) Tyr447 and Tyr 733 mutated to Phe residues, and (2) 7-amino acid peptide (EYHHYNK) being inserted after position 590, as well as adjacent changes.

The present vector may comprise one or more of the following sequences: SEQ ID NO: 9, SEQ ID NO: 10, SEQ ID NO: 11, and SEQ ID NO: 12.

Adenoviruses are described in, e.g., Rosenfeld et al., 1991, Science 252:431-434; Rosenfeld et al., 1992, Cell 68:143-155; Mastrangeli et al., 1993, J. Clin. Invest. 91:225-234; Kozarsky and Wilson, 1993, Curr. Opin. Genetics Develop. 3:499-503; Bout et al., 1994, Human Gene Therapy 5:3-10; PCT Publication No. WO 94/12649; and Wang et al., 1995, Gene Therapy 2:775-783). U.S. Pat. No. 7,244,617.

Greater detail about retroviral vectors is available in Boesen et al., 1994, Biotherapy 6:291-302. In one embodiment, the present expression vector is a lentivirus (including human immunodeficiency virus (HIV)), which is a sub-type of retrovirus.

Plasmids that may be used as the present expression vector include, but are not limited to, pGL3, pCDM8 (Seed, 1987, “An LFA-3 cDNA encodes a phospholipid-linked membrane protein homologous to its receptor CD2”, Nature. 840-842) and pMT2PC (Kaufman et al., 1987, “Translational efficiency of polycistronic mRNAs and their utilization to express heterologous genes in mammalian cells”, EMBO J. 6:187-193). Any suitable plasmid may be used in the present invention.

Certain vectors are capable of autonomous replication in a host cell into which they are introduced (e.g., episomal mammalian vectors). Other vectors (e.g., non-episomal mammalian vectors) are integrated into the genome of a host cell upon introduction into the host cell, and thereby are replicated along with the host genome.

### Gene Therapy

The present expression vectors, compositions and methods may be used in gene therapy. Gene therapy refers to therapy performed by the administration to a subject of an expressed or expressible nucleic acid. Accordingly, the present invention provides for a method for treating or preventing a condition including cardiovascular diseases comprising administering to a patient in need thereof an effective amount of the present expression vector.

Any composition described for administration by gene therapy can also be useful, apart from gene therapy approaches, for in vitro or ex vivo manipulations.

Any of the methods for gene therapy available in the art can be used in accordance with the present invention (See, e.g., Goldspiel et al., 1993, Clinical Pharmacy 12:488-505; Grossman and Wilson, 1993, Curr. Opin. in Genetics and Devel. 3:110-114; Salmons and Gunzberg, 1993, Human Gene Therapy 4:129-141; Morgan and Anderson, 1993, Ann. Rev. Biochem. 62:191-217; Mulligan, 1993, Science 260:926-932; Tolstoshev, 1993, Ann. Rev. Pharmacol. Toxicol. 32:573-596; and Clowes et al., 1994, J. Clin. Invest. 93:644-651; Kiem et al., 1994, Blood 83:1467-1473, each of which is incorporated herein by reference). Gene therapy vectors can be administered to a subject systemically or locally by, for example, intravenous injection (See, e.g., U.S. Pat. No. 5,328,470) or by stereotactic injection (See, e.g., Chen et al., 1994, Proc Natl Acad Sci. 91:3054-57).

A pharmaceutical preparation of the gene therapy vector can comprise a gene therapy vector in an acceptable diluent, or can comprise a slow release matrix in which the gene delivery vehicle is embedded. Alternatively, where the complete gene delivery vector can be produced intact from recombinant cells, the pharmaceutical preparation can include one or more cells which produce the gene delivery system.

Gene therapy involves introducing a gene construct to cells in tissue culture or in vivo. Methods for introduction of polynucleotides of the invention to cells in vitro include, but are not limited to, electroporation, lipofection, calcium phosphate-mediated transfection, and viral infection. Usually, the method of transfer includes the transfer of a selectable marker to the cells. The cells are then placed under selection to isolate those cells that have taken up and are expressing the transferred gene. Those cells are then delivered to a subject. In another embodiment, the polynucleotides of the invention can be introduced into the target tissue as an implant, for example, in a polymer formulation (See, e.g., U.S. Pat. No. 5,702,717). In another embodiment, the polynucleotides of the invention can be targeted to the desired cells or tissues.

An expression vector can be delivered directly into a subject. In one embodiment, the polynucleotides of the invention can be injected directly into the target tissue or cell derivation site. Alternatively, a subject's cells are first transfected with an expression construct in vitro, after which the transfected cells are administered back into the subject (i.e., ex vivo gene therapy). Accordingly, the polynucleotides of the invention can be delivered in vivo or ex vivo to target cells. Several methods have been developed for delivering the polynucleotides of the invention to target cells or target tissues.

In a particular embodiment, a vector is introduced in vivo such that it is taken up by a cell and directs the expression of the therapeutic agent of the invention. Such a vector can remain episomal or can chromosomally integrate.

In a specific embodiment, an expression vector is administered directly in vivo, where the vector is expressed to produce the encoded product. This can be accomplished by any of numerous methods known in the art, e.g., by placing a nucleic acid of the invention in an appropriate expression vector such that, upon administration, the vector becomes intracellular and expresses a therapeutic agent. Such vectors can be internalized by using, for example, a defective or attenuated retroviral vector or other viral vectors that can infect mammalian cells (See, e.g., U.S. Pat. No. 4,980,286).

Alternatively, an expression construct comprising a nucleic acid of the invention can be injected directly into a target tissue as naked DNA. In another embodiment, an expression vector can be introduced intracellularly using microparticle bombardment, for example, by using a Biolistic gene gun (Dupont). In another embodiment, an expression construct comprising a nucleic acid of the invention can be coated with lipids, or cell-surface receptors, or transfecting agents, such that encapsulation in liposomes, microparticles, or microcapsules facilitates access to target tissues and/or entry into target cells. In yet another embodiment, an expression construct comprising a nucleic acid of the invention is linked to a polypeptide that is internalized in a subset of cells or is targeted to a particular cellular compartment. In a further embodiment, the linked polypeptide is a nuclear targeting sequence which targets the vector to the cell nucleus. In another embodiment, the linked polypeptide is a ligand that is internalized by receptor-mediated endocytosis in cells expressing the respective receptor for the ligand (See, e.g., Wu and Wu, 1987, J. Biol. Chem. 262:4429-4432).

In another embodiment, nucleic acid-ligand complexes can be formed such that the ligand comprises a fusogenic viral peptide which disrupts endosomes, thereby allowing the nucleic acid to avoid lysosomal degradation. In another embodiment, a nucleic acid of the invention can be targeted in vivo via a cell-specific receptor resulting in cell-specific uptake and expression (See, e.g., International Patent Publications WO 92/06180, WO 92/22635, WO 92/20316, WO 93/14188, and WO 93/2022. In yet another embodiment, a nucleic acid of the invention is introduced intracellularly and, by homologous recombination, can transiently or stably be incorporated within the host cell DNA, which then allows for its expression, (Koller and Smithies, 1989, Proc Natl Acad Sci. 86:8932-8935; Zijlstra et al., 1989, Nature 342:435-438).

In this embodiment, the expression vector is introduced into a cell prior to administration in vivo of the resulting recombinant cell. Such introduction can be carried out by any method known in the art, including, but not limited to, transfection, electroporation, microinjection, infection with a viral or bacteriophage vector comprising the polynucleotides, cell fusion, chromosome-mediated gene transfer, microcell-mediated gene transfer, and spheroplast fusion. Numerous techniques are known in the art for the introduction of foreign genes into cells (See, e.g., Maniatis et al., 1989; Current Protocols in Molecular Biology, John Wiley & Sons, 2000; Loeffler and Behr, 1993, Meth. Enzymol. 217:599-618; Cohen et al., 1993, Meth. Enzymol. 217:618-644; Cline, 1985, Pharmacol. Ther. 29:69-92) and can be used in accordance with the present invention.

The resulting recombinant cells can be delivered to a subject by various methods known in the art, and the skilled artisan would appreciate appropriate modes of administration. For example, intravenous administration may be the preferred mode of administration for recombinant hematopoietic stem cells. Similarly, the number of recombinant cells to be administered to a subject can be determined by one skilled in the art, and would include a consideration of factors such as the desired effect, the disease state, and the mode of administration.

### Conditions Treated

The present expression vectors, compositions and methods may be used to treat or prevent a cardiovascular disease, such as atherosclerosis, stenosis, restenosis, hypertension, heart failure, left ventricular hypertrophy (LVH), myocardial infarction, acute coronary syndrome, stroke, transient ischemic attack, impaired circulation, heart disease, cholesterol and plaque formation, ischemia, ischemia reperfusion injury, peripheral vascular disease, myocardial infection, cardiac disease (e.g, risk stratification of chest pain and interventional procedures), cardiopulmonary resuscitation, kidney failure, thrombosis (e.g., venous thrombosis, deep vein thrombosis, portal vein thrombosis, renal vein thrombosis, jugular vein thrombosis, cerebral venous sinus thrombosis, arterial thrombosis, etc.), thrombus formation, thrombotic event or complication, Budd-Chiari syndrome, Paget-Schroetter disease, coronary heart disease, coronary artery disease, need for coronary revascularization, peripheral artery disease, a pulmonary circulatory disease, pulmonary embolism, a cerebrovascular disease, cellular proliferation and endothelial dysfunction, graft occlusion or failure, need for or an adverse clinical outcome after peripheral bypass graft surgery, need for or an adverse clinical outcome after coronary artery bypass (CABG) surgery, failure or adverse outcome after angioplasty, internal mammary artery graft failure, vein graft failure, autologous vein grafts, vein graft occlusion, ischaemic diseases, intravascular coagulation, cerebrovascular disease, or any other cardiovascular disease related to obesity or an overweight condition.

Conditions that may also be treated using the present compositions and methods include diseases which are more prominent due to aging or increased inflammation, such as stroke, arthritis, and dementia. Schwarz et al., Identification of differentially expressed genes induced by transient ischemic stroke, Brain Res Mol Brain Res. 2002; 101(1-2):12-22. Simopoulou et al., Lectin-like oxidized low density lipoprotein receptor 1 (LOX-1) expression in human articular chondrocytes. Clin Exp Rheumatol. 2007, 25(4):605-12. Kakinuma et al., Lectin-like oxidized low-density lipoprotein receptor 1 mediates matrix metalloproteinase 3 synthesis enhanced by oxidized low-density lipoprotein in rheumatoid arthritis cartilage, Arthritis Rheum. 2004, 50(11):3495-503. Li et al., The new role of LOX-1 in hypertension induced neuronal apoptosis, Biochem Biophys Res Commun. 2012 Sep. 7; 425(4):735-40.

Non-limiting examples of the conditions the present expression vectors, compositions and methods may be used to treat or prevent include rheumatoid arthritis, adenosine deaminase deficiency, hemophilia, cystic fibrosis, and hyperproliferative disorders (e.g., cancer etc.), bone resorption, osteoporosis, osteoarthritis, periodontitis, hypochlorhydia and neuromyelitis optica, insulin resistance, glucose intolerance, diabetes mellitus, hyperglycemia, hyperlipidemia, dyslipidemia, hypercholesteremia, hypertriglyceridemia, hyperinsulinemia, diabetic dyslipidemia, HIV-related lipodystrophy, peripheral vessel disease, cholesterol gallstones, menstrual abnormalities, infertility, polycystic ovaries, osteoarthritis, sleep apnea, metabolic syndrome (Syndrome X), type II diabetes, diabetic complications including diabetic neuropathy, nephropathy, retinopathy, cataracts, any other asthmatic and pulmonary diseases related to obesity and any other viral infection similar to HIV-1 infection related diseases.

### Pharmaceutical Compositions

The present invention provides for a pharmaceutical composition comprising a therapeutically effective amount of the present expression vector.

The present agents or pharmaceutical compositions may be administered by any route, including, without limitation, oral, transdermal, ocular, intraperitoneal, intravenous, ICV, intracisternal injection or infusion, subcutaneous, implant, sublingual, subcutaneous, intramuscular, intravenous, rectal, mucosal, ophthalmic, intrathecal, intra-articular, intra-arterial, sub-arachinoid, bronchial and lymphatic administration. The present composition may be administered parenterally or systemically.

The pharmaceutical compositions of the present invention can be, e.g., in a solid, semi-solid, or liquid formulation. Intranasal formulation can be delivered as a spray or in a drop; inhalation formulation can be delivered using a nebulizer or similar device; topical formulation may be in the form of gel, ointment, paste, lotion, cream, poultice, cataplasm, plaster, dermal patch aerosol, etc.; transdermal formulation may be administered via a transdermal patch or iontorphoresis. Compositions can also take the form of tablets, pills, capsules, semisolids, powders, sustained release formulations, solutions, emulsions, suspensions, elixirs, aerosols, chewing bars or any other appropriate compositions.

The composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed release bolus, or continuous administration.

To prepare such pharmaceutical compositions, one or more of compound of the present invention may be mixed with a pharmaceutical acceptable excipient, e.g., a carrier, adjuvant and/or diluent, according to conventional pharmaceutical compounding techniques.

Pharmaceutically acceptable carriers that can be used in the present compositions encompass any of the standard pharmaceutical carriers, such as a phosphate buffered saline solution, water, and emulsions, such as an oil/water or water/oil emulsion, and various types of wetting agents. The compositions can additionally contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols. For examples of carriers, stabilizers, preservatives and adjuvants, see Remington's Pharmaceutical Sciences, edited by E. W. Martin (Mack Publishing Company, 18th ed., 1990). Additional excipients, for example sweetening, flavoring and coloring agents, may also be present.

The pharmaceutically acceptable excipient may be selected from the group consisting of fillers, e.g. sugars and/or sugar alcohols, e.g. lactose, sorbitol, mannitol, maltodextrin, etc.; surfactants, e.g. sodium lauryle sulfate, Brij 96 or Tween 80; disintegrants, e.g. sodium starch glycolate, maize starch or derivatives thereof; binder, e.g. povidone, crosspovidone, polyvinylalcohols, hydroxypropylmethylcellulose; lubricants, e.g. stearic acid or its salts; flowability enhancers, e.g. silicium dioxide; sweeteners, e.g. aspartame; and/or colorants. Pharmaceutically acceptable carriers include any and all clinically useful solvents, dispersion media, coatings, antibacterial and antifungal agents, isotonic and absorption delaying agents and the like.

The pharmaceutical composition may contain excipients for modifying, maintaining or preserving, for example, the pH, osmolarity, viscosity, clarity, color, isotonicity, odor, sterility, stability, rate of dissolution or release, adsorption or penetration of the composition. Suitable excipients include, but are not limited to, amino acids (such as glycine, glutamine, asparagine, arginine or lysine); antimicrobials; antioxidants (such as ascorbic acid, sodium sulfite or sodium hydrogen sulfite); buffers (such as borate, bicarbonate, Tris HCl, citrates, phosphates, other organic acids); bulking agents (such as mannitol or glycine), chelating agents (such as ethylenediamine tetraacetic acid (EDTA), ethylene glycol tetraacetic acid (EGTA)); complexing agents (such as caffeine, polyvinylpyrrolidone, beta cyclodextrin or hydroxypropyl beta cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (in one aspect, sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company, 1990).

Oral dosage forms may be tablets, capsules, bars, sachets, granules, syrups and aqueous or oily suspensions. Tablets may be formed form a mixture of the active compounds with fillers, for example calcium phosphate; disintegrating agents, for example maize starch, lubricating agents, for example magnesium stearate; binders, for example microcrystalline cellulose or polyvinylpyrrolidone and other optional ingredients known in the art to permit tabletting the mixture by known methods. Similarly, capsules, for example hard or soft gelatin capsules, containing the active compound, may be prepared by known methods. The contents of the capsule may be formulated using known methods so as to give sustained release of the active compounds. Other dosage forms for oral administration include, for example, aqueous suspensions containing the active compounds in an aqueous medium in the presence of a non-toxic suspending agent such as sodium carboxymethylcellulose, and oily suspensions containing the active compounds in a suitable vegetable oil, for example arachis oil. The active compounds may be formulated into granules with or without additional excipients. The granules may be ingested directly by the patient or they may be added to a suitable liquid carrier (e.g. water) before ingestion. The granules may contain disintegrants, e.g. an effervescent pair formed from an acid and a carbonate or bicarbonate salt to facilitate dispersion in the liquid medium. U.S. Pat. No. 8,263,662.

Intravenous forms include, but are not limited to, bolus and drip injections. Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

Additional compositions include formulations in sustained or controlled delivery, such as using liposome or micelle carriers, bioerodible microparticles or porous beads and depot injections.

The present compound(s) or composition may be administered as a single dose, or as two or more doses (which may or may not contain the same amount of the desired molecule) over time, or as a continuous infusion via implantation device or catheter. The pharmaceutical composition can be prepared in single unit dosage forms.

Appropriate frequency of administration can be determined by one of skill in the art and can be administered once or several times per day (e.g., twice, three, four or five times daily). The compositions of the invention may also be administered once each day or once every other day. The compositions may also be given twice weekly, weekly, monthly, or semi-annually. In the case of acute administration, treatment is typically carried out for periods of hours or days, while chronic treatment can be carried out for weeks, months, or even years. U.S. Pat. No. 8,501,686.

Administration of the compositions of the invention can be carried out using any of several standard methods including, but not limited to, continuous infusion, bolus injection, intermittent infusion, inhalation, or combinations of these methods. For example, one mode of administration that can be used involves continuous intravenous infusion. The infusion of the compositions of the invention can, if desired, be preceded by a bolus injection.

### Kits

The present invention also provides for a kit for use in the treatment or prevention of a cardiovascular disease or other conditions. Kits according to the invention include package(s) (e.g., vessels) comprising agents or compositions of the invention. The kit may include an expression vector of the present invention. The expression vector may be present in unit dosage forms.

Examples of pharmaceutical packaging materials include, but are not limited to, bottles, tubes, inhalers, pumps, bags, vials, containers, syringes, bottles, and any packaging material suitable for a selected formulation and intended mode of administration and treatment.

Kits can contain instructions for administering agents or compositions of the invention to a patient. Kits also can comprise instructions for uses of the present agents or compositions. Kits also can contain labeling or product inserts for the present expression vectors or compositions. The kits also can include buffers for preparing solutions for conducting the methods. The instruction of the kits may state that the expression vector drives disease-specific expression of a therapeutic agent.

Subjects, which may be treated according to the present invention include all animals which may benefit from administration of the agents of the present invention. Such subjects include mammals, preferably humans, but can also be an animal such as dogs and cats, farm animals such as cows, pigs, sheep, horses, goats and the like, and laboratory animals (e.g., rats, mice, guinea pigs, and the like).

The following examples of specific aspects for carrying out the present invention are offered for illustrative purposes only, and are not intended to limit the scope of the present invention in any way.

### Examples

### Example 1 Disease-Limited Interleukin 10 (IL10)-AAV Gene Therapy for Cardiovascular Disease

Many therapeutic genes, such as transforming growth factor beta 1 (TGFβ1) and interleukin 10 (IL10), have some level of adverse effects. The use of a disease-specific promoter may minimize the “downside” of these genes, yet provide enough local expression at the site of disease to effect a cure. The lectin-like oxidized low density lipoprotein receptor 1 (LOX1)(oxidized LDL receptor 1, OLR1) is transcriptionally up-regulated early on in the disease process by a number of cell types, predating and predicting the sites of future disease. In this study, an adeno-associated virus vector (AAV2 backbone), using the AAV8 capsid, and containing the full length LOX1 promoter (LOX1pr; 2.4 kb), was generated and assayed for its ability to express human interleukin 10 (hIL10) for anti-atherosclerotic effect in low density lipoprotein receptor knockout (LDLR KO) mice on high cholesterol diet (HCD). The cytomegalovirus immediate early (CMVpr) promoter was used for comparison in a similarly structured vector. Both AAV2/8.LOX1pr-hIL10 and AAV2/8.CMVpr-hIL10 were found to give statistically equal efficacy in their down-regulation of atherogenesis as measured by aortic systolic blood velocity, aortic cross sectional area, and aortic wall thickness. This is a direct comparison of a constitutive promoter (CMVpr) with a disease-specific promoter (LOX1pr) in a therapeutic context.

### Materials and Methods

### Generation of Recombinant AAV Virus.

We utilized the full length 2.4 kb LOX1 promoter as has been previously characterized (23-31) To generate the AAV/LOX1pr-IL10, the full length Loxl promoter (nt-2402 to +9) was amplified from human 293 cell by PCR using the primers: upstream 5′-ATAATGCATCTTTCTTATTTGGGGGAAG-3′ (SEQ ID NO: 14) and downstream 5′-ATACGCGTACTAAAAATATGTGAGCTTCTG-3′ (SEQ ID NO: 15). Nsi I and Mlu I (underlined) sites were included in the primers to allow easy ligation in front of the hIL10 gene within the gutted AAV2 plasmid d13-97. Construction and generation of AAV/Neo and AAV/CMVpr-hIL10 recombinant virus have been described previously (28, 37, 50). For comparison we utilized the cytomegalovirus immediate early constitutive promoter (CMVpr), which is well studied (33, 34). The virus stocks were generated and titered by dot blot hybridization as described previously (18, 32, 50). The titers were calculated to be about 1×10⁹ encapsidated genomes per ml (eg/ml).

### Animal Treatments.

The low density lipoprotein receptor (LDLR) knockout mouse (B6; 129S7-Ldlr^{tm1Her}/J) was used in these studies, and was purchased from Jackson Laboratories (Bar Harbor, Me., USA). Two groups of male mice weighing 16-20 grams were tail vein injected with AAV2/8.LOX1pr-hIL10, AAV2/8.CMVpr-hIL10, or AAV2/8.SV40pr-Neo virus, each at a titer of 1×10⁹ eg/ml via tail vein injections of 200 uL virus/mouse, followed by two booster injections at an interval of approximately 5 days. High cholesterol diet (HCD) of 4% cholesterol and 10% Coco butter diet (Harlan Teklad, Madison, Wis., USA) was then provided on the day of first injection and continuously maintained for twenty weeks. This high fat level HCD diet was used to ensure the development of atherosclerosis. Another control group included mice fed a normal chow diet. All animals were weighed weekly starting at 16 weeks post-injection.

### High Resolution Ultrasound Imaging

The Vevo 770 High-Resolution Imaging system (Visualsonics, Toronto, Canada) was used for ultrasound imaging, using an RMV 707B transducer, having a center frequency of 30 MHz. Animals were prepared as described earlier (50). Briefly, eight to ten mice from each group were anesthetized with 1.5% isoflurane (Isothesia, Abbot Laboratories, Chicago, USA), with supplemental oxygen and laid supine on a thermostatically heated platform. Their legs were taped to ECG electrodes for cardiac function monitoring. A shaver was used to remove abdominal hair along with a chemical hair remover (Church & Dwight Co, Inc., NJ, USA) and pre-warmed US gel (Medline Industries, Inc., Mundelein, USA) was then spread over the skin as a coupling medium for the transducer. The thoracic/abdominal region of the aorta was visualized, below the aortic arches to the diaphragm. Image acquisition was started in the B-mode, where, a long axis view was used to visualize the length of the aorta. The scanhead probe was then turned 90° for a short-axis view to obtain pictures of the cross-sectional area of the aorta. Individual frames and cine loops (300 frames) were also acquired at all levels of the aorta both in long axis and short axis view and recorded at distances of 1 mm throughout the length of the aorta. The flow velocity, orientation of the abdominal aorta on ultrasound, was accomplished by tilting the platform and the head of mouse down with the transducer probe towards the feet and tail of the mouse. The described positioning ensured that the Doppler angle was less than 60° for accurate measurements of blood flow velocity in the pulse-wave Doppler (PW) mode within the aorta. Measurements and data analysis was performed off-line on the longitudinal and transverse images using the customized version of Vevo770 Analytical Software. It took approximately 25-30 minutes to carry out the complete imaging for each mouse.

### Measurement of Plasma Cholesterol

The Veterans Animal Laboratory (VAMU) determined the plasma levels of total cholesterol for the animal groups, measured by VetScan VS2 (ABAXIS, Union City, Calif.).

hIL10 Gene Expression Analysis Using Real-Time Quantitative Reverse Transcription PCR (QRT-PCR)

Six mice from each group were sacrificed and total aortic RNA was extracted with TRIzol extraction (Invitrogen Carlsbad, Calif.) according to the manufacturer's instructions. cDNA was generated using random hexamer primers and RNase H-reverse transcriptase (Invitrogen, Carlsbad, Calif.). QRT-PCR was then performed using the Applied Biosystems Fast 7900HT real-time PCR system (Applied Biosystems, Foster City, Calif.) as described (33). We designed qRT-PCR specific primers for analyzing human IL10 using Probe-Finder (http://www.roche-applied-science.com) web-based software from Human and Mouse Universal ProbeLibrary from Roche Applied Science. The results were further analyzed using SDS 2.3 relative quantification (RQ) manager software. The comparative threshold cycles (Ct) values were normalized for the βactin reference gene and then compared with a calibrator by the 2^{-ΔΔct} method.

AAV2/8 delivers hIL10

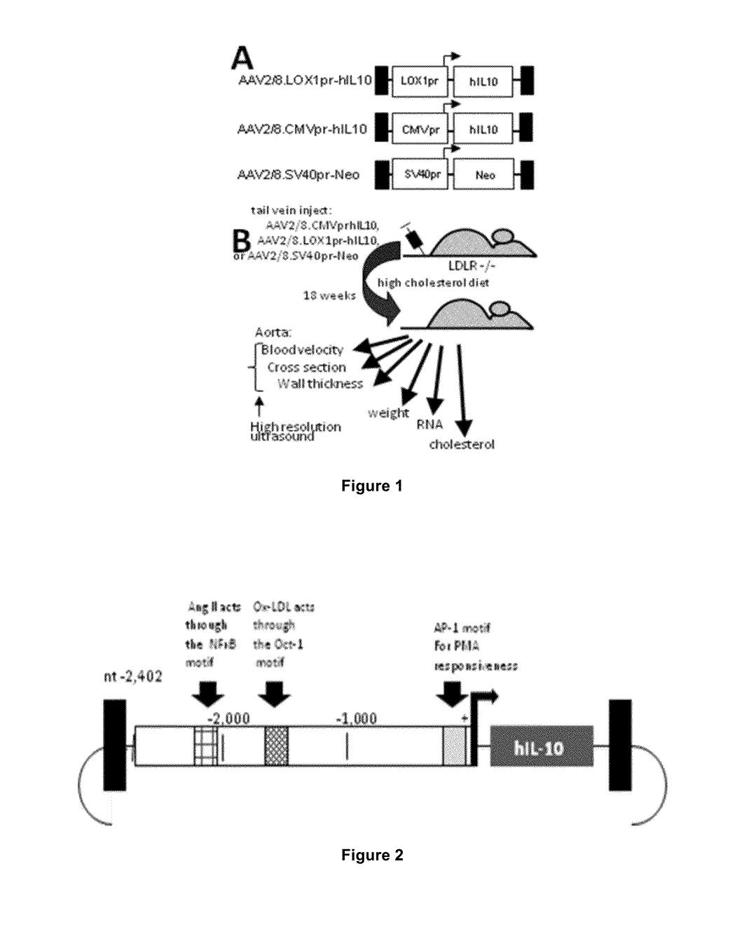

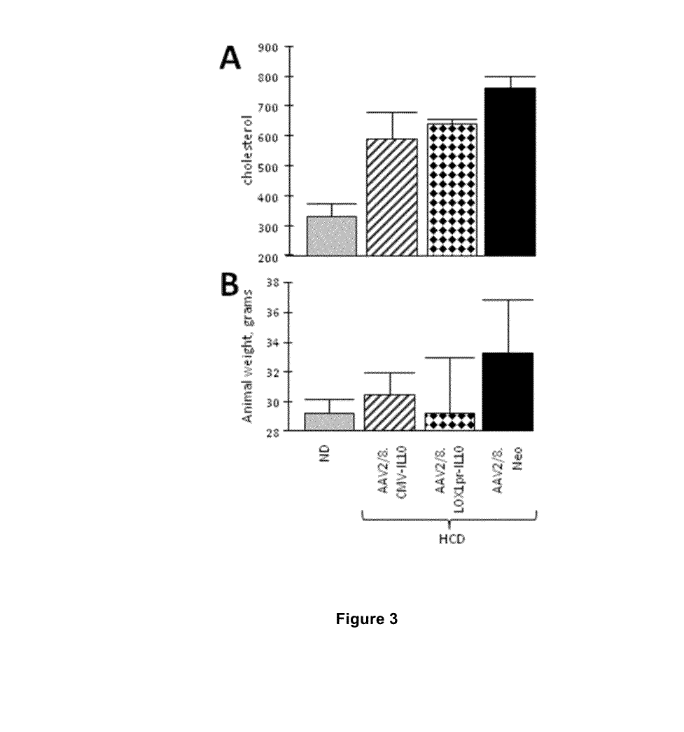

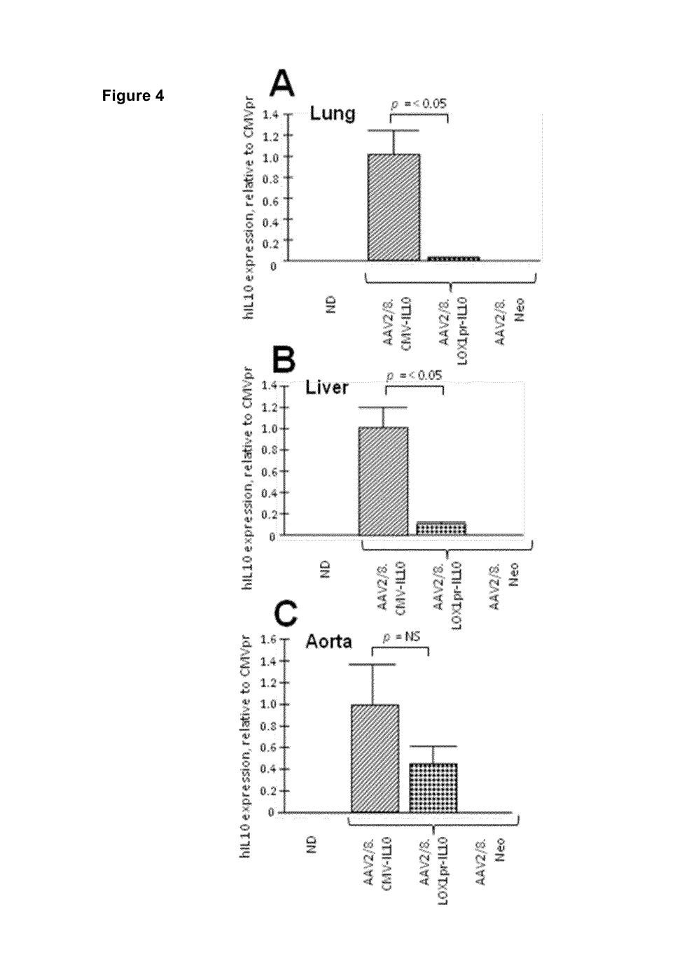

In order to investigate the use of a disease-specific promoter to limit the possibility of adverse reactions to systemic IL-10 expression (18-20), the efficacy of AAV2/8.LOX1pr-IL10 was compared to AAV2/8.CMVpr-IL10 delivery in LDLR KO mice and then placed on high cholesterol diet (HCD). An AAV/Neomycin resistance gene (Neo) vector (AAV2/8.SV40pr-Neo) was also used as a non-therapeutic, null control. Vector structures are shown in FIG. 1A and the overall experimental scheme in FIG. 1B. A more detailed view of the AAV.LOX1pr-hIL10 vector plasmid is shown in FIG. 2. At the time of harvest, a portion of mice were sacrificed to determine the success of gene delivery by analyzing hIL10 mRNA expression in the aorta using qRT-PCR analysis. This analysis utilized mRNA isolated from 6 mouse aortas from each group, harvested at week 20. Representative results for LOX1pr- and CMVpr-hIL10-treated mice are shown in FIG. 3, and both vectors were observed to be highly expressed in aortas at a level of appropriately 0.1-0.2% that of (3-actin. Moreover, while the expression by CMVpr and LOX1pr were statistically similar, it is apparent that the LOX1pr expression trended lower, less than 50% than that of CMVpr.

Both Therapeutic CMVpr-hIL10 and LOX1pr-hIL10 Transgene Delivery Inhibits Aortic Blood Flow Velocity with Equal Efficacy.

Having demonstrated significant gene delivery/expression for both CMVpr-hIL10 or LOX1pr-hIL10, we then studied the effects of the transgene. FIG. 3A shows that CMVpr-hIL10 and LOX1pr-hIL10-transgene-HCD-treated animals had similar levels of total cholesterol levels, and were comparable to Neo-treated HCD-fed animals, however hIL-10 treated animals trended slightly lower as has been seen previously. Regarding animal weights, all treatments were statistically the same as normal diet (ND) controls but trended slightly lower in both hIL10-treated animal groups (FIG. 3B).

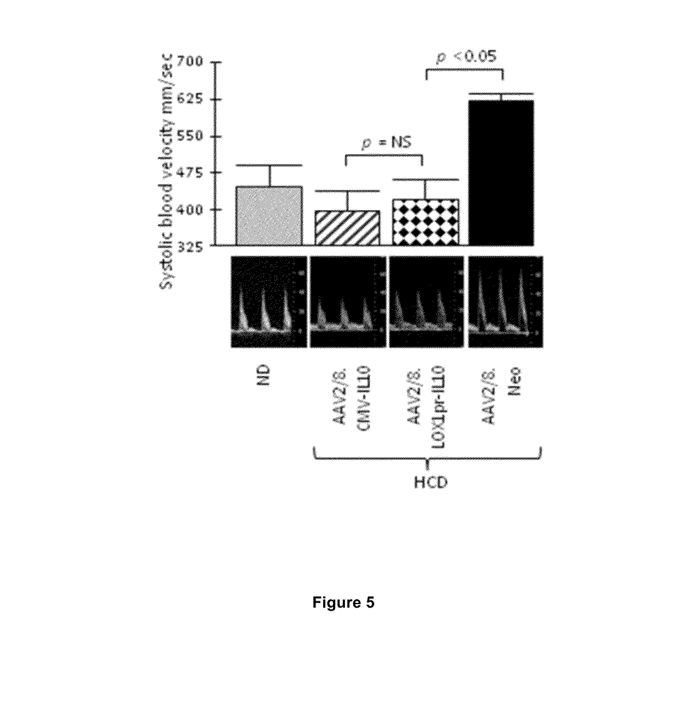

We analyzed arterial blood flow velocity as an easy technique to calculate the extent of atherosclerosis in the mouse groups. The systolic blood velocity in the thoracic/abdominal region of the aorta was quantified by high resolution ultrasound (HRUS) imaging system Vevo 770 with measurements taken on eight-to-ten animals. FIG. 5 shows the quantified systolic blood velocity from five separate measurements on each animal. It can be seen that Neo-HCD-treated animals displayed significantly higher blood flow velocities in their aortas, consistent with a constricted aortic lumen. Additionally, both CMVpr- and LOX1pr-driven gene therapies (hIL-10) on HCD had markedly lower flow velocity than the Neo-HCD-treated group, and very similar to that of the ND fed control animals. Thus, as suggested by lower blood velocity, both CMVpr- and LOX1pr-treatments displayed an anti-atherosclerotic effect and were statistically similar in their degree efficacy.

Both Therapeutic CMVpr-hIL10 and LOX1pr-hIL10 Gene Delivery Inhibits Aortic Structural Changes Associated with Atherosclerosis with Equal Efficacy.

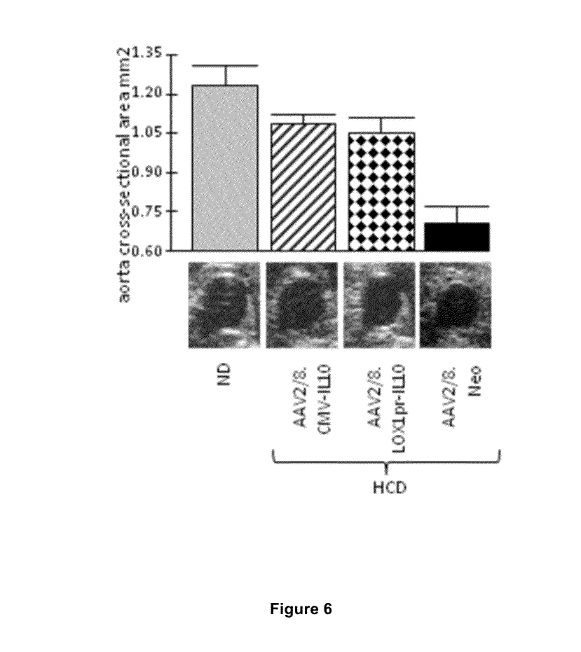

We then analyzed structural changes within the aortas of the various experimental groups by first measuring the aortic cross sectional area. Multiple measurements were taken from eight-to-ten animals in each group. The measurements were made in the same thoracic/abdominal site (see Materials and Methods). FIG. 6 shows the quantified results for the thoracic/abdominal region of the aorta. It can be seen that, the AAV/Neo-HCD-treated positive control animals had the smallest cross sectional lumen area, being consistent with higher atherosclerosis. In contrast, the two therapeutic treatments, both LOX1pr-hIL10-HCD and CMVpr-IL10, both on HCD, had much larger lumens than Neo-treated-HCD controls and this difference was statistically significant. The ND group trended to have the largest lumens.

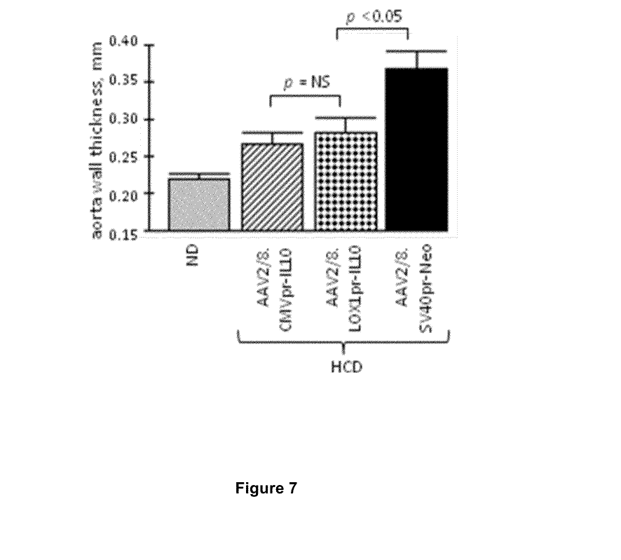

The wall thickness of the thoracic region of the aorta was also measured. FIG. 7 shows the quantified results for the thoracic region of the aorta. The Neo-HCD-treated animals displayed the thickest aortic walls, while both CMVpr- and LOX1pr-hIL10 vector-HCD-treatments were efficacious=<0.05), and were statistically similar to each other (p=NS. As expected, the ND group had the thinnest aortic walls. Thus all three measurements by HRUS (aortic systolic blood velocity, lumen size, and wall thickness) showed that the LOX1pr-driven vector was equally efficacious to the CMVpr version.

### Discussion

Here we demonstrate that an AAV2/8.LOX1pr-hIL10 vector was effective in inhibiting atherogenesis in LDLR KO mice on HCD, and was statistically equal to the inhibition derived from an AAV2/8.CMV-IL10 vector delivery. This is a direct comparison of a disease-specific promoter to a general expression promoter for determination of level of efficacy. The cytomegalovirus immediate early promoter is perhaps the most used promoter in gene delivery experiments. Thus this study establishes that disease-specific promoters are still able to express therapeutic transgenes to efficacious levels. It has been shown that the 2.4 kb LOX1 promoter fragment has significant activity and responsiveness to disease-associated stimuli as does the wt LOX-1 promoter. Normally the basal level LOX1 promoter activity is very low (44-48). In the future, we will also utilize a non-secreted marker gene to observe the activity of the vector bound disease-specific promoter. It is likely that OxLDL plays a role as we enhanced this agent with the HCD, but other stimulations are also possible, such as angiotensin II. The LOX1 promoter has been recently reviewed (35).

Moreover, in addition to LOX-1 as an atherogenesis-active promoter, there are a multiplicity of genes that are similarly up-regulated. DNA microarray experiments certainly drive this point home with many of genes being found regulated (52-56). The milieu of cells present with the artery wall during atherogenesis also changes over time (57-59). This issue should be taken into account when considering the exact disease-specific promoter to use, its strength, timing and possible cell preference. In normal arteries low monocyte populations exist within the arterial wall, while over time with the activation of the endothelium and recruitment of additional monocytes this number dramatically increases. Yet, our AAV vectors are systemically delivered before HCD-initiated disease. Thus transduction of the general monocyte population is likely taking place at some level, including some which will ultimately migrate into the arterial wall. Related to this issue, here, we have utilized a model of atherosclerosis in which atherogenesis is an active, ongoing process, induced by HCD, starting from a null standpoint. Another relevant model is the observation of regression of an established plaque.

We have studied the therapeutic use of AAV/IL10 and downstream STAT3 to limit atherogenesis in the LDLR KO mouse. This series of studies (37,59,60), along with those of others (36,37), has shown that IL10, and the IL10 signal transduction pathway, is beneficial for limiting atherogenesis by gene therapy. The first study, with IL10 being expressed from AAV2's own p5 promoter, demonstrated that IL10 was effective in inhibiting plaque development in the LDLR KO mouse on HCD (18). In the second installment we demonstrated that STAT3, a gene through which IL10 signal transduction works, also confers anti-atherogenesis effect (59). STAT3 gene delivery could be useful as a substitute gene for IL10, hopefully with less adverse reactions than IL10. The third study examined IL10-plus-STAT3 dual gene delivery, studying the hypothesis that two genes in the same anti-inflammatory signal transduction pathway might act synergistically to limit atherosclerosis (60). Finally, going back to the use of original IL10 gene alone, here we demonstrate that AAV2/8.LOX1pr-IL10 delivery was equally efficacious as AAV2/8.CMVpr-IL10 delivery in limiting the development of atherogenesis. The LOX1pr structure in FIG. 2 shows that the DNA elements needed for Ox-LDL and AngII responsiveness represent only a small part of the full 2.4 kb promoter. This suggests some condensation of the LOX1 promoter might be undertaken to make this disease-specific promoter shorter and more useful for a wider variety of transgenes. This is important as the packaging of AAV genomes becomes more problematic as the size of the genome rises above 4.7 kb (61). However, additional disease-specific promoters and alterations to IL10 or other downstream genes besides STAT3, may be studied. This study also solidifies the usefulness and utility of adeno-associated virus vectors for cardiovascular gene therapy (37-40,59,60) as well as other diseases (34-37).

### Example 2 Disease-Limited Interleukin 10 (IL10)-AAV Gene Therapy for Cardiovascular Disease

### Modification of the AAV Vector

For the safe design of vectors carrying such powerful genes we have pioneered the “disease-specific promoter” gene expression approach in gene therapy. A disease-specific promoter will limit the expression and “adverse reactions” of IL10, yet provides adequate expression at the site of disease to give treatment. The LOX1 gene is transcriptionally up-regulated early on in CVD (cardiovascular disease) in a number of cell types, including smooth muscle cells, predating and predicting the sites of future CVD.

We tested this disease-limited gene therapy hypothesis by studying an adeno-associated virus vector (AAV2 backbone), using the AAV8 capsid, and containing the full length LOX1 promoter (LOX1pr; 2.4 kb) driving expression of the human (h)IL10, for anti-atherosclerotic effect in low density lipoprotein receptor knockout (LDLR KO) mice on high cholesterol diet (HCD). We compared AAV2/8.LOX1pr-hIL10, with the LOX1pr driving hIL10 expression, to AAV2/8.CMVpr-hIL10. CMVpr is a strong constitutive promoter used for comparison (on all the time, positive control). The LOX1pr vector gave statistically equal efficacy to the CMVpr vector in down-regulating atherogenesis as measured by aortic cross sectional area, aortic wall thickness, and aortic systolic blood velocity, yet overall IL10 expression was significantly lower with the LOX1pr. In summary, the disease-specific LOX1pr gives therapeutic expression of IL10, but while maintaining overall lower IL10 expression.

With a disease-limited AAV2-LOX1pr-hIL10 DNA vector backbone capable of inhibiting atherosclerosis in a well respected animal model, our goal is to generate improved AAV-technologies for enhanced delivery of the AAV2-LOX1pr-IL10 vector backbone, and to test its safety in large animal models. In this study, we have three tasks. In Task 1, we will generate two specific improvements to the above proven therapeutic technology generating a more effective AAV2/8(cvd1). The improvements include EYH motif and tyrosine modifications in the AAV8 capsid, such as tyrosine substitution and EYH insertion into the AAV8 capsid gene. LOX1pr-IL10 vector, which will then be tested in explanted human arteries for improved gene delivery. In Task 2, this same vector will be analyzed for the titer needed for efficacy against HCD-induced atherosclerosis in a mouse model (e.g., C57BL/6 LDLR KO mice), and in Task 3, for the titer needed for efficacy in a rabbit model (e.g., familial hypercholesterolemic WHHL rabbits). The completion of these tasks will allow us to move on to toxicology analysis of our anti-atherosclerotic gene therapy agent before finally going to PHASE I clinical trials in patients.

### Specific Aims

We will modify the AAV8 capsid by two modifications and demonstrate its improved gene delivery capabilities and then assay the efficacy of disease-specific LOX1pr-hIL10 gene cassette by gene delivery into two animal models of atherosclerosis, the LDLR KO mouse and WHHL rabbit.

We will carry out the following tasks.
1) Generate single and dual modified (combined) tyrosine-minus, EYH-positive AAV8 capsid and test improvements in gene delivery in human placental umbilical cord blood vessels.
2) Determine titer of AAV2/8(cvd1).LOX1pr-IL10 vector needed for efficacy against HCD-induced atherosclerosis in LDLR KO mice.
3) Determine titer of AAV2/8(cvd1).LOX1pr-IL10 vector needed for efficacy against HCD-induced atherosclerosis in WHHL rabbits.
   Results may include the following:
1) Improve gene delivery into umbilical cord blood vessels smooth muscle cells at least about 3 fold above wild type AAV8 capsid vectors.
2) Inhibit HCD-induced atherosclerosis in LDLR KO mice by at least 70%.
3) Inhibit HCD-induced atherosclerosis in WHHL rabbits by at least 70%.

### Research Strategy

The targets for arterial gene delivery include the smooth muscle cells which express LOX-1.

### Disease-Limited IL10 Expression Using the LOX1 Promoter for Moderate Expression at the Site of Disease.

Using the LOX1 transcriptional promoter (LOX1pr) as a disease-specific promoter for expressing therapeutic genes to counter atherogenesis, we compared AAV2/8.LOX1pr-hIL10 gene delivery to AAV2/8.CMVpr-hIL10 gene delivery and find that both provide statistically similar efficacy.

The first technology will be to improve the AAV serotype 8 (AAV8) capsid protein with two modifications to increase gene delivery into arteries. These are amino acid tyrosine modifications, for capsid survival, and EYH peptide insertion, for very high frequency infection of smooth muscle cells.

### Approach

### Additional Advantages of AAV Vectors for CVD-Targeting Gene Therapy Treatments.

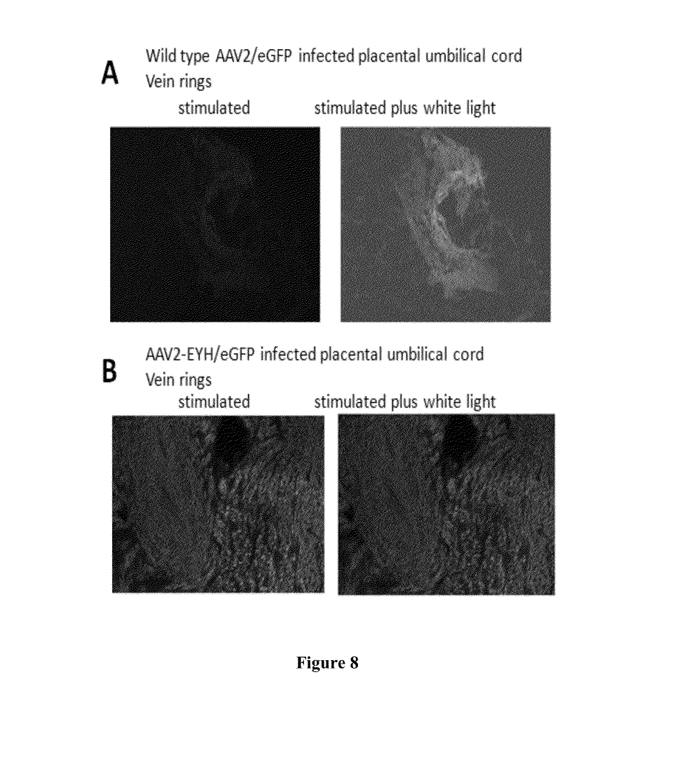

We and others (28-34) have shown that gene expression by AAV can be stably maintained for many months. Moreover, AAV is efficiently taken up by cardiovascular tissues (28-34,27,29). Richter et al. (50) showed efficient transduction in carotid arteries and that smooth muscle cells (SMCs) were the primary target. We found similar results (see FIG. 8), with uptake in the liver, lungs, kidneys, and blood vessels, via a single intravenous injection. In fact, AAV has the advantage of both high safety and long term expression.

### Modification of AAV by Insertion of EYH Improves Smooth Muscle Cell Transduction.

We modified the AAV2 capsid by inserting a peptide, EYHHYNK (EYH), which promotes infection of human vascular smooth muscle cells (VSMCs) in culture (66-69). We repeated the general AAV capsid modification by inserting the EYH peptide sequence, as a DNA sequence, into the AAV capsid gene at amino acid (AA) position 588. We then assayed for its ability to transduce human VSMCs within human placental umbilical cord vein rings in culture. As shown in FIG. 8, we observed that the EYH modification of the AAV2 capsid enhanced the transduction of the VSMCs.

This enhancement with the AAV2/EYH modification motivated us to employ this AAV capsid in the proposed research instead of AAV8, which we had used most recently. We also have additional capsid modifications that we may substitute for AAV2/EYH if they prove superior. For example, a series of tyrosine mutations within the AAV2 capsid gene which, when replaced by phenylalanine, was shown to allow for 10- to 50-fold higher transduction (69). The most important of these is at amino acid (aa) 730 (69). While AAV2 is the most used vector and, historically, the first AAV serotype to be used for gene delivery, we have used AAV8 extensively for arterial gene delivery (60-62). Our goals in this phase will be to translate both of these modifications into AAV8, and generate an AAV8/Y730F/EYH foundation vector. These modifications should give us a highly effective cardiovascular gene therapy vector from which we will deliver our therapeutic transgene, LOX1pr-IL10.

AAV2/8.LOX1pr-hIL10 Demonstrates that Disease-Limited Expression of IL10 Provides Efficacy Against Atherogenesis, Yet with Significantly Lower Overall Expression than when Using the CMVpr.

This study solidifies the importance of the disease-specific gene therapy approach. This is also important as most gene therapy approaches use constitutive promoters which express genes in an “everywhere, all the time” approach. This is dangerous.

### Research Design and Methods

Task 1 is the generation of a new capsid, protein coat, a modification of AAV8, which gives higher transduction of smooth muscle cells in arteries. Our goal will be to improve gene delivery into umbilical cord blood vessels at least 5 fold above wild type AAV8 capsid vectors. This will give us a new, improved modified AAV8 vector for gene delivery into arteries. This improved CVD gene delivery vector will be referred to as AAV2/8(cvd1).LOX1pr-IL10. Task 2 is to determine titer of AAV2/8(cvd1).LOX1pr-IL10 vector, generated in Task 1, needed for efficacy against HCD-induced atherosclerosis in LDLR KO mice. Task 2's findings will determine the level of virus needed to test in the Phase II toxicology studies, as a prelude before advancing to clinical studies. Task 3 is to determine titer of AAV2/8(cvd1).LOX1pr-IL10 vector, generated in Task 1, needed for efficacy against HCD-induced atherosclerosis in WHHL rabbits.

Task 1: Modification of AAV8 by Replacement of Specific Tyrosines and Insertion of EYH Peptide.

Task 1 encompass two modifications of the AAV8 capsid. One modification will be the insertion of the EYH peptide, which improves infection to human vascular smooth muscle cells (see FIG. 8). Smooth muscle cells are in abundance in the normal aorta, as well in the advanced atheroma. Moreover, LOX-1 is up-regulated in arterial smooth muscle cells by a variety of insults such as oxidized low density lipoprotein and pro-inflammatory cytokines (73-74). The other will be the removal of certain tyrosine amino acids which are targeted by the cells protease systems for premature AAV capsid degradation. Thus our goals here will be transfer both of these valuable modifications into AAV8, and generate an AAV8/Y730F/EYH foundation vector. These modifications should give us a highly effective cardiovascular gene therapy vector from which we will be able to deliver our therapeutic transgene, LOX1pr-IL10. The actual DNA sequences of the AAV vector will NOT change. Thus the disease-limited expression of the AAV/LOX1pr-IL10 vector will function specifically as shown in Example 1. The only change will be in that the capsid modifications will result in a higher rate of delivery of these AAV vectors into the arteries.

We will modify the AAV8 capsid, as we have the AAV2 capsid, by inserting the EYHHYNK (EYH) peptide, which promotes binding to and infection of human vascular smooth muscle cells (VSMCs) in culture (68-70). We will utilize GenScript to generate the modifications within the AAV8 helper plasmid pDG8. While the original work was done with AAV2 infection of cultured human VSMCs, we repeated the general AAV capsid modification in AAV8 by inserting the EYH peptide sequence, as a DNA sequence, into the AAV capsid gene at amino acid (AA) position 590 (75). We then assayed for its ability to transduce human VSMCs within human placental umbilical cord vein rings in culture. As shown in FIG. 8, we observed that EYH enhanced the transduction of the VSMCs.

Modification of AAV8 by Insertion of EYH and by Replacing Specific Tyrosines with F/phenylalanine.

We will then modify the AAV8 capsid to carry both of the modifications using GenScriptand, again, test these, two modifications together for improved ability to transduce human VSMCs within human placental umbilical cord vein rings in culture.

Modification of AAV8 by Replacing Specific Tyrosines with F/phenylalanine.

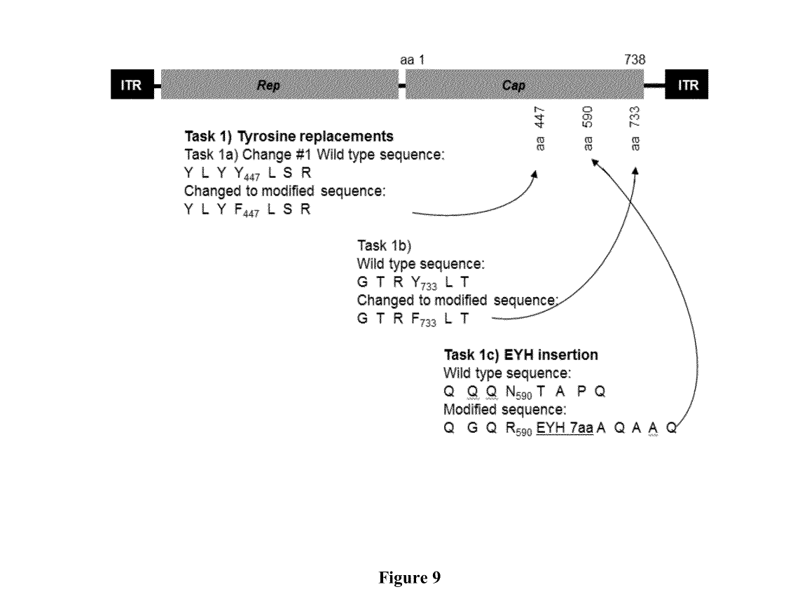

We will also have additional capsid modifications (Genscript) that we will carry out in addition to AAV8/EYH. A series of tyrosine mutations within the AAV2 capsid gene which, when replaced by phenylalanine, has been reported to allow for 10- to 50-fold higher transduction (69). The most important of these substitutions is at amino acid (aa) 444 and 730 (69). The analogous positions to aa444 and 730 of AAV2 in the AAV8 capsid protein are aa447 and 733 (see FIG. 9) (76). We will then generate AAV8/Y447F/Y733F/590-EYH virus which include the enhanced green florescence protein (eGFP) and assay for its ability to transduce human VSMCs within human placental umbilical cord vein rings in culture.

Hereafter AAV2/8(cvd1) refers to the modified AAV2/8-Y730E-EYH capsid derived from Task 1.

Task 2: Test Efficacy in LDLR KO Mice.

The goal in Task 2 is to determine the amount, titer of, AAV2/8(cvd1).LOX1pr-IL10 (Kcardio-1) which is needed to give efficacy. We will use the LDLR KO mouse model to determine this efficacious titer. Three different doses, 10⁸, 10⁹, and 10¹⁰ eg (encapsidated genomes), of the AAV2/8(cvd1).LOX1pr-IL10. Our goal will be to inhibit atherosclerosis in the LDLR KO mouse model by 70% when placed on a high fat high cholesterol diet (HCD). The information on the virus amount needed for efficacy will then be utilized in the toxicology studies of Phase2. Methods are described in more detail in our previous publications (26-31,33,35,48,48,70-72).

Viral Genome Construction and AAV Production.

We have utilized standard protocols described by our laboratory to develop the AAV delivery vector AAV2/8(cvd1).LOX1pr-IL10 which is shown in FIG. 2.

Justification of Animal Numbers.

Power analysis of from our earlier experiments indicates we will need animal group numbers of 12.

Animals, Tissues, and Transgene Expression Analysis.

C57BL/6 LDLR KO mice (18-20 grams) in each of the seven treatment groups will include: AAV2/8(cvd1) refers to the modified AAV2/Y730F/EYH vector capsid derived from Task 1.

1) AAV2/8(cvd1)/Neo-normal diet (ND),
2) AAV2/8(cvd1)/Neo-HCD,
3) AAV2/8(cvd1)/empty (no gene) -HCD,
4) AAV2/8(cvd1).LOX1pr-IL10 at 10⁸ eg -HCD,
5) AAV2/8(cvd1).LOX1pr-IL10 at 10⁹ eg -HCD,
6) AAV2/8(cvd1).LOX1pr-IL10 at 10¹⁰ eg -HCD, and
7) ND ctrl mice (12 animals each).
Neo- and IL10-gene treated animals will be fed a high-cholesterol diet (HCD) consisting of 4% cholesterol and 10% cocoa butter (Harlan Teklad, Madison, Wis.) on the day of the first injection. Mice fed normal chow diets will be included as controls. Animals will be weighed weekly.

Analysis of Markers and Cytokines in the Aorta by Quantitative Real-Time Reverse Transcriptase-PCR (qRT-PCR).

This will be done by standard methodologies. The genes to be analyzed include macrophage markers such as CD68, CD36, ITGAM, and EMR. We expect these to be significantly down when AAV/IL10 is delivered. Other genes to be studied include CD4, CD8, CD14, CD25, CD56, interleukin (IL)-2, interferon (IFN)-γ, IL-4, IL-7, IL-10, IL-17, transforming growth factor (TGF)-β1 and β-actin (housekeeping control.

Analysis of Markers and Cytokines by Immunohistochemistry.

Standard methods will be utilized to visualize and measure the respective targets described (28,29,54,64,63). In particular, macrophage markers such as CD68, CD36, ITGAM, and EMR will be studied.

Analysis of Atherogenesis by Oil Red O Staining.

This will be done by standard methodologies as we have done with Sudan IV.

Analysis of Atherosclerosis by Histologic Cross-Section.

Harvested aortas will be fixed in formalin. Ten sections will be placed on slides and analyzed by imaging software (Image Pro Plus, Media Cybernetics) to quantify plaque formation.

High Resolution Ultrasound (HRUS) Imaging.

Ultrasound imaging will be performed with a Vevo 770 High-Resolution Imaging system (Visualsonics, Toronto, Canada) and a RMV 707B transducer with a center frequency of 30 MHz will be done by standard methodologies (27,54,63,64,72-91).

Measurement of Plasma Cholesterol.

Plasma levels of total cholesterol in mice from respective treatment groups will be measured by VetScan VS2 (ABAXIS, Union City) at the Veterans Animal Laboratory (VAMU).

Analysis of Atherogenesis by Direct Visualization.

Whole dissected aortas will be fixed in 10% buffered formalin and inspected under a dissecting microscope, photographed, and photodigitally analyzed by Image J software (ImageJ, US National Institutes of Health, Bethesda, Md.).

Statistical Analysis.

Statistical analyses will be performed by nonparametric ANOVA analysis with the Statistical Program for Social Sciences version 11.5 for Windows (SPSS Inc, Chicago). If differences are detected between means, then the Newman-Keuls test will be used for multiple comparisons. Differences will considered significant for P<0.05.

Task 3: Test Efficacy in WHHL Rabbits.

The goal in Task 3 is to determine the amount, titer of, AAV2/8(cvd1).LOX1pr-IL10 (Kcardio-1) which is needed to give efficacy. We will use a second animal model to determine efficacious titer, the Watanabe heritable hyperlipidemic (WHHL) rabbit (LDL receptor deficient). Three different doses, 10¹⁰, 10¹¹, and 10¹² eg (encapsidated genomes), of the AAV2/8(cvd1).LOX1pr-IL10 virus. Our goal will be to inhibit atherosclerosis in the WHHL rabbit model by 70% when placed on a high fat high cholesterol diet (HCD). WHHL rabbits will be infected at 2 months old before the development of atherosclerosis (HCD is not needed) and analyzed at 6 months when atherosclerosis is expected to cover about 35% of the aortic surface. (The information on the virus amount needed for efficacy will then utilized at a 10×/weight, ten fold level in the toxicology studies in Phase II. The animal groups will be: 1) AAV2/8(cvd1)/Neo-normal diet (ND), 2) AAV2/8(cvd1)/empty, 3) AAV2/8 cvd1).LOX1pr-IL10 at 10¹⁰ eg, 4) AAV2/8 cvd.1).LOX1pr-IL10 at 10¹¹ eg, 5) AAV2/8 cvd1).LOX1pr-IL 10 at 10¹² eg, and 6) ND ctrl rabbits (5 animals each). All experimental studies will be done similar to Task 2 with the LDLR KO mice, with the assays and readings being scaled up for the increased size of rabbits (1,500-2000 grams) over that of mice (18-24 grams).

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

### CITATIONS

1) Hermonat P L, and Muzyczka N. Use of adeno-associated virus as a mammalian DNA cloning vector: transduction of neomycin resistance into mammalian tissue culture cells. Proc. Natl. Acad. Sci. U.S.A. 81:6466-6470, 1984.
2) Hermonat P L. The first adeno-associated virus gene transfer experiment, 1983. In press Human Gene Therapy, 2014.
3) Buchlis, G, Podsakoff G M, Radu A, Hawk S M, Flake A W, Mingozzi F, High K A. Factor IX expression in skeletal muscle of a severe hemophilia B patient 10 years after AAV-mediated gene transfer. Blood, 2012 Mar. 29; 119(13):3038-41.
4) Zhu H, Cao M, Chriva-Internati M, Hermonat P L. Comparison of efficacy of human interleukin 10, expressed from the disease-specific LOX1 or constitutive cytomegalovirus promoters, against atherosclerosis in mice using adeno-associated virus 2/8 delivery. in press PLoS ONE, 2014.
5) Zhu H, Cao M, Figueroa J A, Cobos E, Uretsky B F, Chiriva-Internati M, Hermonat, P L. (2014) AAV2/8-hSMAD3 gene delivery attenuates aortic atherogenesis, enhances Th2 response, without inducing COL1A2/2A1 and CTGF (fibrosis) in LDLR-KO mice on high cholesterol diet. In press J Translational Medicine.
6) Zhu H, Cao M, Straub K D, Hermonat P L. (2013) Systemic delivery of thiol-specific antioxidant hPRDX6 gene by AAV2/8 inhibits atherogenesis in LDLR KO mice on HCD. In press Genetic Syndromes and Gene Therapy 7) Damdindorj L, Kaman S, Ota A, Hossain E, Konishi Y, Hosokawa Y, Konishi H. A comparative analysis of constitutive promoters located in adeno-associated viral vectors. PLoS One. 2014 Aug. 29; 9(8):e106472
8) Filippi C M, von Herrath M G. IL-10 and the resolution of infections. High levels of IL10 are associated with increased viral, bacterial, and fungal infections, as well as cancer. J Pathol 214: 224-230, 2008.
9) Zobel, Katrin. Martus, Peter. Pletz, Mathias W. Ewig, Santiago. Prediger, Michael. Welte, Tobias. Buhling, Frank. Interleukin 6, lipopolysaccharide-binding protein and interleukin 10 in the prediction of risk and etiologic patterns in patients with community-acquired pneumonia: results from the German competence network CAPNETZ. BMC Pulmonary Medicine. 12:6, 2012.
10) Clemons K V. Grunig G. Sobel R A. Mirels L F. Rennick D M. Stevens D A. Role of IL-10 in invasive aspergillosis: increased resistance of IL-10 gene knockout mice to lethal systemic aspergillosis. Clinical & Experimental Immunology. 122(2):186-91, 2000.
11) Brooks, D. G., A. M. Lee, H. Elsaesser, D. B. McGovern, and M. B. Oldstone. IL-10 blockade facilitates DNA vaccine-induced T cell responses and enhances clearance of persistent virus infection. J. Exp. Med. 205:533-541, 2008.
12) Brooks, D. G., M. J. Trifilo, K. H. Edelmann, L. Teyton, D. B. McGovern, and M. B. Oldstone. Interleukin-10 determines viral clearance or persistence in vivo. Nat. Med. 12:1301-1309, 2006.
13) Ejrnaes, M., C. M. Filippi, M. M. Martinic, E. M. Ling, L. M. Togher, S. Crotty, and M. G. von Herrath. Resolution of a chronic viral infection after interleukin-10 receptor blockade. J. Exp. Med. 203:2461-2472, 2006.
14) Zeni E, Mazzetti L, Miotto D, Lo Cascio N, Maestrelli P, et al. Macrophage expression of interleukin-10 is a prognostic factoir in nonsmall cell lung cancer. Eur Respir J 30: 627-632, 2007.
15) Maris, C. H., C. P. Chappell, and J. Jacob. Interleukin-10 plays an early role in generating virus-specific T cell anergy. BMC Immunol. 8:8. 2007.
16) Herfarth H, Scholmerich J. IL-10 therapy in Crohn's disease: at the crossroads. Gut 50:146-147.
17) Hermonat P L, Zhou H, Cao M, Mehta J L. LOX-1 transcription. Cardiovasc Drugs Ther., 2012.
18) Flotte T R, Afione S A, Solow R, Drumm M L, Markakis D, Guggino W B, Zeitlin P L, Carter B J. Expression of the cystic fibrosis transmembrane conductance regulator from a novel adeno-associated virus promoter. J Biol Chem 268: 3781-3790, 1993.
19) Haberman R P, McCown T J, Samulski R J. Novel transcriptional regulatory signals in the adeno-associated virus terminal repeat A/D junction element. J Virol 74: 8731-8739, 2000.
20) Libby, P, Ridker, P M, Maseri, A. (2002) Inflammation and Atherosclerosis. Circulation. 105: 1135-1143.
21) Libby, P, Ridker, P M, Hansson, G K. Inflammation in atherosclerosis. (2009) J Amer Coll Card 54:2129-2138.
22) Ludewig B, Laman J D. (2004) The in and out of monocytes in atherosclerotic plaques: Balancing inflammation through migration. Proc Natl Acad Sci USA. 101: 11529-11530.
23) Woollard K J, Geissmann F. (2010) Monocytes in atherosclerosis: subsets and functions. Nat Rev Cardiol. 7: 77-86.
24) Lacolley P, Regnault V, Nicoletti A, Li Z, Michel J B. The vascular smooth muscle cell in arterial pathology: a cell that can take on multiple roles. Cardiovasc Res 95: 194-2004, 2012.
25) Rebsen S S M, Doevendans P A F M, van Eys G J J M. Regulation and characteristics of vascular smooth muscle cell phenotypic diversity. Neth Heart J. 15: 100-108, 2007.
26) Liu Y, Li D, Chen J, Xie J, Bandyopadhyay S, et al. (2006) Inhibition of atherogenesis in LDLR knockout mice by systemic delivery of adeno-associated virus type 2-hIL-10. Atherosclerosis 188: 19-27.
27) Khan J A, Cao M, Kang B Y, Liu Y, Mehta J L, Hermonat P L. AAV/IL-10 plus STAT3 anti-inflammatory gene delivery lowers atherosclerosis in LDLR KO mice, but without increased benefir. Int J Vasc Med 2012: 52435, 2012.
28) Chen S, Kapturczak M H, Wasserfall C, Glushakova O Y, Campbell-Thompson M, et al. (2006) Interleukin 10 attenuates neointimal proliferation and inflammation in aortic allografts by a heme oxygenase-dependent pathway. Proc Natl Acad Sci 102: 7251-7256.
29) Yoshioka T, Okada T, Maeda Y, Ikeda U, Shimpo M, et al. (2004) Adeno-associated virus vector-mediated interleukin-10 gene transfer inhibits atherosclerosis in apolipoprotein E-deficient mice. Gene Therapy 11: 1772-1779.
30) Sun J, Li X, Feng H, Gu H, Blair T, Li J, Soriano S, Meng Y, Zhang F, Feng Q, Yang X. Magnetic resonance imaging of bone marrow-cell mediated interleukin-10 gene therapy of atherosclerosis. PloS One 6(9): e24529, 2011
31) Han X, Kitamoto S, Wang H, Boisvert W A. Interleukin-10 overexpression in macrophages suppresses atherosclerosis in hyperlipidemic mice. FASEB J 24: 2869-2880, 2010.
32) Li, D., et al. Suppression of Atherogenesis by Delivery of TGF$1ACT Using Adeno-Associated Virus Type 2 in LDLR Knockout Mice. Biochem. Biophys. Res. Commun. 344: 701-707, 2006.
33) Dandapat A, Hermonat P L, Mehta J L. Over-expression of TGFbeta(1) by adeno-associated virus type 2 vector protects myocardium from ischemia-reperfusion injury. Gene Ther. 15: 415-23, 2008.
34) Letterio J J, Roberts A B. (1998) Regulation of immune responses by TGF-beta. Annu Rev Immunol 16:137-161.
35) de Vries J E. (1995) Immunosuppressive and anti-inflammatory properties of interleukin 10. Ann Med. 27:537-41.
36) Byfield S D, Roberts A B. (2004) Lateral signaling enhances TGF-beta response complexity. Trends Cell Biol 14:107-111.
37) Attisano L, Wrana J L. (2002) Signal Transduction by the TGF-beta Superfamily. Science 296:1646-1647.
38) Kopp J B, Factor V M, Mozes M, Nagy P, Sanderson N, et al. (1996) Transgenic mice with increased plasma levels of TGF-beta 1 develop progressive renal disease. Lab Invest 74: 991-1003.
39) Gressner A M, Weiskirchen R, Breitkopf K, Dooley S. (2002) Roles of TGF-beta in hepatic fibrosis. Front Biosci 7: 793-807.
40) Filippi C M, von Herrath M G. IL-10 and the resolution of infections. High levels of IL10 are associated with increased viral, bacterial, and fungal infections, as well as cancer. J Pathol 214: 224-230, 2008.
41) Zobel, Katrin. Martus, Peter. Pletz, Mathias W. Ewig, Santiago. Prediger, Michael. Welte, Tobias. Buhling, Frank. Interleukin 6, lipopolysaccharide-binding protein and interleukin 10 in the prediction of risk and etiologic patterns in patients with community-acquired pneumonia: results from the German competence network CAPNETZ. BMC Pulmonary Medicine. 12:6, 2012.
42) Clemons K V. Grunig G. Sobel R A. Mirels L F. Rennick D M. Stevens D A. Role of IL-10 in invasive aspergillosis: increased resistance of IL-10 gene knockout mice to lethal systemic aspergillosis. Clinical & Experimental Immunology. 122(2):186-91, 2000 Nov.
43) Brooks, D. G., A. M. Lee, H. Elsaesser, D. B. McGavern, and M. B. Oldstone.
2008. IL-10 blockade facilitates DNA vaccine-induced T cell responses and enhances clearance of persistent virus infection. J. Exp. Med. 205:533-541
44) Brooks, D. G., M. J. Trifilo, K. H. Edelmann, L. Teyton, D. B. McGavern, and M. B. Oldstone. 2006. Interleukin-10 determines viral clearance or persistence in vivo. Nat. Med. 12:1301-1309.
45) Ejrnaes, M., C. M. Filippi, M. M. Martinic, E. M. Ling, L. M. Togher, S. Crotty, and M. G. von Herrath. 2006. Resolution of a chronic viral infection after interleukin-10 receptor blockade. J. Exp. Med. 203:2461-2472
46) Maris, C. H., C. P. Chappell, and J. Jacob. 2007. Interleukin-10 plays an early role in generating virus-specific T cell anergy. BMC Immunol. 8:8.
47) Herfarth H, Scholmerich J. (2002) IL-10 therapy in Crohn's disease: at the crossroads Gut 50:146-147.
48) Asadullah K, Sterry W, Volk H D. (2003) Interleukin-10 Therapy—Review of a New Approach Pharmacological Reviews 55: 241-269.
49) Hermonat P L, Muzyczka N. (1984) Use of adeno-associated virus as a mammalian DNA cloning vector: transduction of neomycin resistance into mammalian tissue culture cells. Proc Natl Acad Sci USA 81: 6466-6470.
50) Tratschin J D, West M H, Sandbank T, Carter B J. (1984) A human parvovirus, adeno-associated virus, as a eukaryotic vector: transient expression and encapsidation of the prokaryotic gene for chloramphenicol acetyltransferase, Molec Cell Biol 4: 2072-2081.
51) Hermonat P, Labow, L M, R. Wright, Berns K I, Muzyczka N. (1984) Genetics of adeno-associated virus: isolation and preliminary characterization of mutants in adeno-associated virus type 2. J Virol 51: 329-339.
52) Liu Y, Chiriva-Internati M, Grizzi F, Salati E, Roman J J, Lim S, et al. (2001) Rapid induction of cytotoxic T cell response against cervical cancer cells by human papillomavirus type 16 E6 antigen gene delivery into human dendritic cells by an adeno-associated virus vector. Can Gene Ther 8: 948-957.
53) Chiriva-Internati M, Liu Y, Weidanz J A, Grizzi F, You H, Zhou W, et al. (2003)
Testing recombinant adeno-associated virus-gene loading of dendritic cells for generating potent cytotoxic T lymphocytes against a prototype self-antigen, multiple myeloma HM1.24. Blood. 102: 3100-3107.
54) You C X, Shi M, Liu Y, Cao M, Luo R C, et al. (2012) AAV2/IL-12 gene delivery into dendritic cells (D C) enhances CTL stimulation above other IL-12 applications: evidence for IL-12 intracrine activity in D C. Oncoimm 2012: 1:847-855.
55) Jiang H., Pierce G F, Ozelo M C, de Paula E V, Vargas J A, et al. (2006) Evidence of multiyear factor IX expression by AAV-mediated gene transfer to skeletal muscle in an individual with severe hemophilia B. Molec Ther 24: 452-455.
56) Wettergren E E, Gussing F, Quintino L, Lundberg C. (2012) Novel disease-specific promoters for use in gene therapy for Parkinson's disease. Neurosci Lett 530: 29-34
57) Kim H A, Mahato R I, Lee M. (2009) Hypoxia-specific gene expression for ischemic disease gene therapy Adv Drug Del Rev 61: 614-622
58) Sawamura T, Kume N, Aoyama T, Morlaki H, Hoshikawa H, et al. (1997) An endothelial receptor for oxidized low-density lipoprotein. Nature 386:73-77.
59) Oka K, Sawamura T, Kikuta K, Itokawa S, Kume N, et al. (1998) Lectin-like oxidized low density lipoprotein receptor 1 mediates phagocytosis of aged/apoptotic cells in endothelial cells. Proc Natl Acad Sci USA 95: 9535-9540.
60) Mehta J L, Li D. (1998) Identification and autoregulation of receptor for OX-LDL in cultured human coronary artery endothelial cells. Biochem Biophys Res Commun 248: 511-514.
61) Kakutani M, Masaki T, Sawamura T. (2000) A platelet-endothelium interaction mediated by lectin-like oxidized low-density lipoprotein receptor-1. Proc Natl Acad Sci USA. 97: 360-364.
62) Ogura S, Kakino A, Sato Y, Fujita Y, Iwamoto S, Otsui K, et al. (2009) LOX-1: the multifunctional receptor underlying cardiovascular dysfunction. Circ J 73:1993-1999.
63) Aoyama T, Sawamura T, Furutani Y, Matsuoka R, Yoshida M C, Fujiwara H, et al. (1999) Structure and chromosomal assignment of the human lectin-like oxidized low-density-lipoprotein receptor-1 (LOX-1) gene. Biochem J. 339:177-184.
64) Khan, J A, Cao M, Kang B Y, Liu Y, Mehta J L, Hermonat P L. (2010) AAV/hSTAT3-gene delivery lowers aortic inflammatory cell infiltration in LDLR KO mice on high cholesterol diet. Atherosclerosis 213: 59-66.
65) Richter M, Iwata A, Nyhuis J, Nitta Y, Miller A D, Halbert C L, Allen M D. Adeno-associated virus vector transduction of vascular smooth muscle cells in vivo. Physiol Genomics 2: 117-127, 2000.
66) Li Q, Muruve D A. Molecular basis of the inflammatory response to adenovirus vectors. Gene Ther 10: 935-940, 2003.
67) Jooss K, Chirmule N. Immunity to adenovirus and adeno-associated viral vectors: implications for gene therapy. Gene Ther 10: 9550963, 2003.
68) Work L M, Nicklin S A, Brain NJR, Dishart K L, von Seggern D J, et al. Development of efficient viral vectors selective for vascular smooth muscle cells. Mol Ther 9: 198-208, 2004.
69) Zhong L, Li B, Mah C S, Govindasamy L, Agbandje-McKenna M, Cooper M, et al. Next generation of adeno-associated virus vectors: point mutations in tyrosines lead to high-efficiency transduction at lower doses. Proc Natl Acad Sci USA 105: 7827-7832, 2008.
70) Wang Z, Zhu T, Qiao C, Zhou L, Wang B, et al. Adeno-associated virus serotype 8 efficiently delivers genes to muscle and heart. Nat Biotechnol 23: 321-328, 2005.
71) Efficacy and safety of adeno-associated viral vectors based on serotype 8 and 9 vs lentiviral vectors for hemophilia B gene therapy. Vandendriessche T, Thorrez L, Acosta-Sanchez A, Petrus I, Wang L, et al. Efficacy and safety of adeno-associated viral vectors based on serotype 8 and 9 vs lentiviral vectors for hemophilia B gene therapy. J Thromb Haemost 5: 16-24, 2007.
72) Hermonat P L, Quirk J G, Bishop B M, Han L. Packaging capacity of adeno-associated virus and the potential for wild type-plus AAV gene therapy vectors. FEBS Letters 407:78-84, 1997.
73) Holhagel 0, Luechtenborg B, Stolle K, Lorkowski S, Eschert H, Plenz G, Robenek H. Proinflammatory cytokines regulate LOX-1 expression in vascular smooth muscle cells. Arterioscler Tromb Vasc Biol 24: 1789-1795, 2004.
74) Sun Y, Chen X. Ox-LDL-induced LOX-1 expression in vascular smooth muscle cells: role of reactive oxygen species. Fundam Clin Pharmacol 25: 572-579, 2011.
75) Michelfelder S, Varadi K, Raupp C, Hunger A, Korbelin J, Pahrmann C, et al. Peptide ligands incorporated into the threefold spike capsid domain to re-direct gene transduction of AAV8 and AAV9 in vivo. PlosOne 6: e23101, 2011.
76) Kay C N, Ryals R C, Aslanidi G V, Min S H, Ruan Q, Sun J, et al. Targeting photoreceptors via intravitreal delivery using novel capsid-mutated AAV vectors. PlosOne 8: e62097, 2013.
77) Shiomi M, Koike T, Ito T. Contribution of the WHHL rabbit, an animal model of familial hypercholesterolemia, to elucidation of the anti-atherosclerotic effects of statins. Atherosclerosis 231: 39-47,2013.
78) Liu Y, Li D, Chen J, Xie J, Bandyopadhyay S, et al. (2006) Inhibition of atherogenesis in LDLR knockout mice by systemic delivery of adeno-associated virus type 2-hIL-10. Atherosclerosis 188: 19-27.
79) Chen J, Liu Y, Liu H, Hermonat P L, Mehta J L. (2006) Lectin-like oxidized low-density lipoprotein receptor-1 (LOX-1) transcriptional regulation by Oct-1 in human endothelial cells: implications for atherosclerosis. Biochemical J 393: 255-265.
80) Chen J, Liu Y, Liu H, Hermonat P L, Mehta J L. (2006) Molecular dissection of angiotensin II-activated human LOX-1 promoter. Arterioscler Thromb Vasc Biol. 26:1163-1168.
81) Qin J Y, Zhang L, Clift K L, Hulur I, Xiang A P, et al. (2010) Systematic comparison of constitutive promoters and the doxycyline-inducible promoter. PLOSone 5: e10611
82) Zarrin A A, Malkin L, Fong I, Luk K D, Ghose A, et al. (1999) Comparison of CMV, RSV, SV40 viral and V lambda 1 cellular promoters in B and T lymphoid and non-lymphoid cell lines. Biochim Biophys Acta 1446: 135-139.
83) Faber B C, Cleutjens K B, Niessen R L, Aarts P L, Boon W, et al. (2001) Identification of genes potentially involved in rupture of human atherosclerotic plaques. Circ Res 89: 547-554.
84) Armstrong P J, Johanning J M, Calton Jr W C, Delatore J R, Franklin D P, et al. (2002) Differential gene expression in human abdominal aorta: aneurysmal versus occlusive disease. J Vasc Surg 35: 346-355.
85) Hiltunen M O, TuomistoBrasen J H, Rissanen T T, et al. (2002) Changes in gene expression in atherosclerotic plaques analyzed using DNA array. Atherosclerosis 165: 23-32.
86) Martinet W, Schrijvers D M, De Meyer G R, Thielemans J, Knaapen M W, et al. (2002) Gene expression profiling of apoptosis-related genes in human atherosclerosis: upregulation of death-associated protein kinase. Arterioscler Thromb Vasc Biol 22: 2023-2029.
87) Woodside K J, Hernandez A, Smith F W, Xue X Y, Hu M, et al. (2003) Differential gene expression in primary and recurrent carotid stenosis. Biochem Biophys Res Commun 302: 509-514.
88) Stary H C. (2000) Natural History and Histological Classification of Atherosclerotic Lesions: An Update, Arterioscler Thromb Vasc Biol 20: 1177-1178.
89) Stone G W, Maehara A, Lansky A J, de Bruyne B, Cristea E, et al. (2011) A prospective natural-history study of coronary atherosclerosis. N Engl J Med 364: 226-235.
90) Cao M, Khan J A, Kang B Y, Mehta J L, Hermonat P L. (2012) Dual AAV/IL-10 plus STAT3 anti-inflammatory gene delivery lowers atherosclerosis in LDLR KO mice, but without increased benefit. Int J Vasc Med. 2012: 524235.
91) Hermonat P L, Quirk J G, Bishop B M, Han L. (1997) Packaging capacity of adeno-associated virus and the potential for wild type-plus AAV gene therapy vectors. FEBS Letters 407:78-84.

SEQUENCE LISTINGS

```
SEQ ID NO: 1
LOX1 promoter: 2.4kb (derived from GenBank Accession No. AH00781.1)
atgaggccca cctacattat gcagcgaaat ctactttcct ctgctgatta
atatgttaat ctcatttaaa agcatcctgt ctgggaaccc catgacccag tcaagaagac
acagaaggcc agcacggtgg ctcacggctt gtaatcccag cactgtggga ggctgaggca
ggcggatcac ttgaggacag gagttcaaga ccagcctggc caacatggtg aaaccctgtc
tctactgaaa atacaaaaca attagccggg aggggtggcg ggcacctgta atcccagcta
ctcaggaggc tgaggcagga gaattgcttg aacccaggag gcggaggttg cagtgagccg
agatcatgcc attgcaaccc accctgggta acaagggcga aactccatct caaaaacaaa
caaacaaaca aacaaacaaa caaaacacat gaaattcacc atcacaggag ttattgcttg
gtgggtatag agtttcagtt ggggaagatg aaaagttctg gagatggatg gtggtgatgg
ctgcacaaca atgtaaatgt acttaatgcc actgaacagt acgcctaaaa atggttgaaa
tggtaaacac tttatgttct ataatattca tggcatccaa aataactgag tcactgtaga
aatgataatg tgcccttttc agtgtgatat cgtttcagta ttttttaatt cttttttttt
ttttttttaa agggagtctc gctctgttgc ccaggctgga gtacagtggc acaatctcag
ctcactgaaa atgaaaagca ttttcatttt ccacctccca aatagctggg attataggct
tgcaaccagg cccagctaac ttttgtattt ttagtggtga cagggtttca acaggttggc
caggctggtc tcgaactcct gacctcagtg atccaccccc ccctcagctt cccaaagtgc
tgagattaca ggcatgagcc actgcacctg gccaactttt tcaatttgtt ctcaaatact
ctgcacataa taaaaggaat ataggaaaca gggaggagga gggaaatgga atcctaatta
ggctgtcaat attggtaatt tttcctaata gtttcctata tgifitattt atgtatttat
ttttccattc ttcctttgtg tattcttttc tttgcacttt tttttcctgg agtaatttaa
agtaaatgcc agatatcata tcattttgct tataaatact tccttcagaa gacatctcta
atagatatat atctggttag gaggttgtca agggaaaata gaaaagtatt ctatcagtaa
atacatatag acatacttat gggtctctca tgtaagcgtc tttgtgacat gagtcaatca
tggattttac tgaaatacat tatttgatgg gtaattcaac tgtgctgaat attagtacta
tactaataat tattatttat tggcttctta ttatgctcca agtacaaagc ttgtcatttt
taaccagcgt gtcatttgaa ttcctatagc aacgtgacat ttttcacaaa cctagtgtaa
aaaagcttat tatttttacc cagcatgtca tttgaattcc tataacaatg ttatcaggta
gacattatcc ctaatttcca gacatgtcat ttgaattcct ataacaatgt tatcaggtag
acattatccc taatttccag acaaagacat agattcaaag tattaagatt tcccaagatt
gtagactaaa ggaaagtact gaaactagct ctttctgcat tcaaagtgct ctctgctgct
acgttacact ggcactgcat gaactagtag ccccagataa attctaaaat cacccaggac
acaccatcgc tggaaatggg agccatatgt gattcacccc aaaattctta gatccattgc
cagataaaac agtgataatc aaaagagaat atatctgatt cataaaagac accatataga
aacagtctgc taatgaaatt agcaaacaaa cattggaatc aaatattttg gcccataatt
ctggcacatt ttttacaaat gtagtgtgac ttactctctt tgaatttcag tttctgtctc
tgaagagtgg gtacaatatc tcctctgatg ctcatgaaaa atagttttcc ctttcataaa
ttacttagcg aaatatcctg aaacaccttc agaatcacca ctttctccac ctgcaataca
cataactcaa gaatttgcgt cagcgaactt cccaatatga agcaaagcct ctccttcctc
ctaccaatga ttgagccatt cttctattag ataacagtag ctatttaaat acttctgcag
aagctcacat atttttagtt tgttgaagtt cgtgactgct tcactctctc attcttagct
tgaatttgga aatgactttt gatgacctaa
SEQ ID NO: 2
Human IL10 coding (nucleotide) sequence (derived from GenBank Accession No.
NM 000572)
aaagaaggca
tgcacagctc agcactgctc tgttgcctgg tcctcctgac tggggtgagg gccagcccag
gccagggcac ccagtctgag aacagctgca cccacttccc aggcaacctg cctaacatgc
ttcgagatct ccgagatgcc ttcagcagag tgaagacttt ctttcaaatg aaggatcagc
tggacaactt gttgttaaag gagtccttgc tggaggactt taagggttac ctgggttgcc
aagccttgtc tgagatgatc cagttttacc tggaggaggt gatgccccaa gctgagaacc
aagacccaga catcaaggcg catgtgaact ccctggggga gaacctgaag accctcaggc
tgaggctacg gcgctgtcat cgatttcttc cctgtgaaaa caagagcaag gccgtggagc
aggtgaagaa tgcctttaat aagctccaag
SEQ ID NOs: 3 and 4
SEQ ID NO: 3: AAV8 capsid coding (nucleotide) sequence with Tyr447 and Tyr 733
mutated to Phe residues (bolded and in capital letters)
SEQ ID NO: 4: AAV8 capsid amino acid sequence with Tyr447 and Tyr 733 mutated to
Phe residues (bolded and in capital letters)

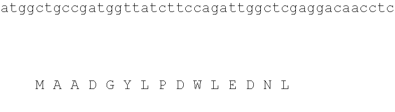

tctgagggcattcgcgagtggtgggcgctgaaacctggagccccg
        S E G I R E W W A L K P G A P
aagcccaaagccaaccagcaaaagcaggacgacggccggggtctg
        K P K A N Q Q K Q D D G R G L
gtgcttcctggctacaagtacctcggacccttcaacggactcgac
        V L P G Y K Y L G P F N G L D
aagggggagcccgtcaacgcggcggacgcagcggccctcgagcac
        K G E P V N A A D A A A L E H
gacaaggcctacgaccagcagctgcaggcgggtgacaatccgtac
        D K A Y D Q Q L Q A G D N P Y
ctgcggtataaccacgccgacgccgagtttcaggagcgtctgcaa
        L R Y N H A D A E F Q E R L Q
gaagatacgtcttttgggggcaacctcgggcgagcagtcttccag
        E D T S F G G N L G R A V F Q
gccaagaagcgggttctcgaacctctcggtctggttgaggaaggc
        A K K R V L E P L G L V E E G
gctaagacggctcctggaaagaagagaccggtagagccatcaccc
        A K T A P G K K R P V E P S P
cagcgttctccagactcctctacgggcatcggcaagaaaggccaa
        Q R S P D S S T G I G K K G Q
cagcccgccagaaaaagactcaattttggtcagactggcgactca
        Q P A R K R L N F G Q T G D S
gagtcagttccagaccctcaacctctcggagaacctccagcagcg
        E S V P D P Q P L G E P P A A

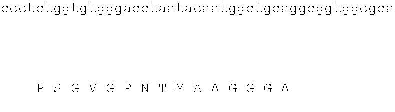

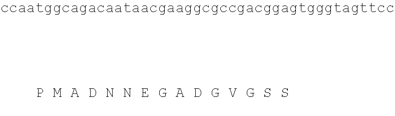


tcgggaaattggcattgcgattccacatggctgggcgacagagtc
        S G N W H C D S T W L G D R V
atcaccaccagcacccgaacctgggccctgcccacctacaacaac
        I T T S T R T W A L P T Y N N
cacctctacaagcaaatctccaacgggacatcgggaggagccacc
        H L Y K Q I S N G T S G G A T
aacgacaacacctacttcggctacagcaccccctgggggtatttt
        N D N T Y F G Y S T P W G Y F
gactttaacagattccactgccacttttcaccacgtgactggcag
        D F N R F H C H F S P R D W Q
cgactcatcaacaacaactggggattccggcccaagagactcagc
        R L I N N N W G F R P K R L S
ttcaagctcttcaacatccaggtcaaggaggtcacgcagaatgaa
        F K L F N I Q V K E V T Q N E
ggcaccaagaccatcgccaataacctcaccagcaccatccaggtg
        G T K T I A N N L T S T I Q V
tttacggactcggagtaccagctgccgtacgttctcggctctgcc
        F T D S E Y Q L P Y V L G S A

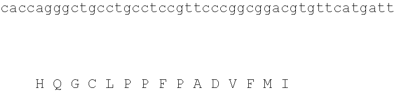

ccccagtacggctacctaacactcaacaacggtagtcaggccgtg
        P Q Y G Y L T L N N G S Q A V

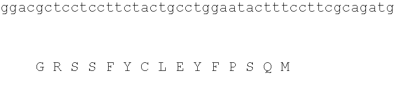

ctgagaaccggcaacaacttccagtttacttacaccttcgaggac
        L R T G N N F Q F T Y T F E D
gtgcctttccacagcagctacgcccacagccagagcttggaccgg
        V P F H S S Y A H S Q S L D R

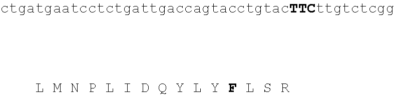

actcaaacaacaggaggcacggcaaatacgcagactctgggcttc
        T Q T T G G T A N T Q T L G F

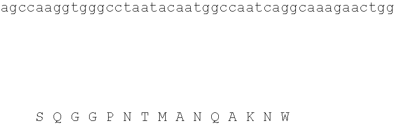

ctgccaggaccctgttaccgccaacaacgcgtctcaacgacaacc
        L P G P C Y R Q Q R V S T T T
gggcaaaacaacaatagcaactttgcctggactgctgggaccaaa
        G Q N N N S N F A W T A G T K
taccatctgaatggaagaaattcattggctaatcctggcatcgct
        Y H L N G R N S L A N P G I A

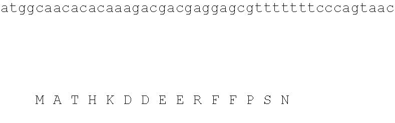

gggatcctgatttttggcaaacaaaatgctgccagagacaatgcg
        G I L I F G K Q N A A R D N A

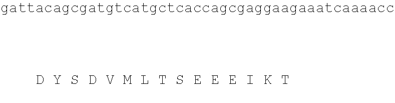

actaaccctgtggctacagaggaatacggtatcgtggcagataac
        T N P V A T E E Y G I V A D N
ttgcagcagcaaaacacggctcctcaaattggaactgtcaacagc
        L Q Q Q N T A P Q I G T V N S

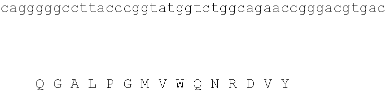

ctgcagggtcccatctgggccaagattcctcacacggacggcaac
        L Q G P I W A K I P H T D G N

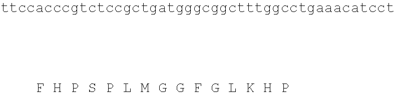

ccgcctcagatcctgatcaagaacacgcctgtacctgcggatcct
        P P Q I L I K N T P V P A D P
ccgaccaccttcaaccagtcaaagctgaactctttcatcacgcaa
        P T T F N Q S K L N S F I T Q
tacagcaccggacaggtcagcgtggaaattgaatgggagctgcag
        Y S T G Q V S V E I E W E L Q
aaggaaaacagcaagcgctggaaccccgagatccagtacacctcc
        K E N S K R W N P E I Q Y T S
aactactacaaatctacaagtgtggactttgctgttaatacagaa
        N Y Y K S T S V D F A V N T E

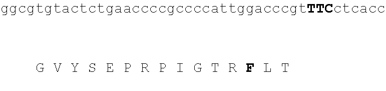

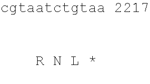


SEQ ID NOs: 5 and 6
SEQ ID NO: 5: AAV8 capsid coding (nucleotide) sequence with EYH modification
(bolded and in capital letters) showing 7 additional amino acids (EYHHYNK) being
inserted after position 590, as well as adjacent changes
SEQ ID NO: 6: AAV8 capsid amino acid sequence with EYH modification (bolded and
in capital letters) showing 7 additional amino acids (EYHHYNK) being inserted after
position 590, as well as adjacent changes

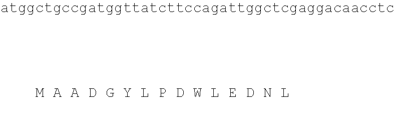

tctgagggcattcgcgagtggtgggcgctgaaacctggagccccg
        S E G I R E W W A L K P G A P
aagcccaaagccaaccagcaaaagcaggacgacggccggggtctg
        K P K A N Q Q K Q D D G R G L
gtgcttcctggctacaagtacctcggacccttcaacggactcgac
        V L P G Y K Y L G P F N G L D
aagggggagcccgtcaacgcggcggacgcagcggccctcgagcac
        K G E P V N A A D A A A L E H
gacaaggcctacgaccagcagctgcaggcgggtgacaatccgtac
        D K A Y D Q Q L Q A G D N P Y
ctgcggtataaccacgccgacgccgagtttcaggagcgtctgcaa
        L R Y N H A D A E F Q E R L Q
gaagatacgtcttttgggggcaacctcgggcgagcagtcttccag
        E D T S F G G N L G R A V F Q
gccaagaagcgggttctcgaacctctcggtctggttgaggaaggc
        A K K R V L E P L G L V E E G
gctaagacggctcctggaaagaagagaccggtagagccatcaccc
        A K T A P G K K R P V E P S P
cagcgttctccagactcctctacgggcatcggcaagaaaggccaa
        Q R S P D S S T G I G K K G Q
cagcccgccagaaaaagactcaattttggtcagactggcgactca
        Q P A R K R L N F G Q T G D S
gagtcagttccagaccctcaacctctcggagaacctccagcagcg
        E S V P D P Q P L G E P P A A

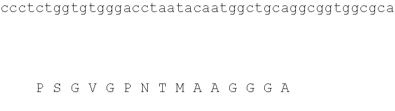

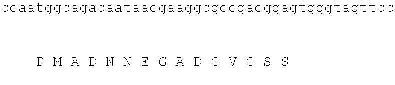


tcgggaaattggcattgcgattccacatggctgggcgacagagtc
        S G N W H C D S T W L G D R V
atcaccaccagcacccgaacctgggccctgcccacctacaacaac
        I T T S T R T W A L P T Y N N
cacctctacaagcaaatctccaacgggacatcgggaggagccacc
        H L Y K Q I S N G T S G G A T
aacgacaacacctacttcggctacagcaccccctgggggtatttt
        N D N T Y F G Y S T P W G Y F
gactttaacagattccactgccacttttcaccacgtgactggcag
        D F N R F H C H F S P R D W Q
cgactcatcaacaacaactggggattccggcccaagagactcagc
        R L I N N N W G F R P K R L S
ttcaagctcttcaacatccaggtcaaggaggtcacgcagaatgaa
        F K L F N I Q V K E V T Q N E
ggcaccaagaccatcgccaataacctcaccagcaccatccaggtg
        G T K T I A N N L T S T I Q V
tttacggactcggagtaccagctgccgtacgttctcggctctgcc
        F T D S E Y Q L P Y V L G S A

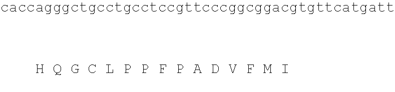

ccccagtacggctacctaacactcaacaacggtagtcaggccgtg
        P Q Y G Y L T L N N G S Q A V

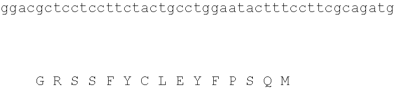

ctgagaaccggcaacaacttccagtttacttacaccttcgaggac
        L R T G N N F Q F T Y T F E D
gtgcctttccacagcagctacgcccacagccagagcttggaccgg
        V P F H S S Y A H S Q S L D R

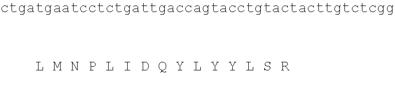

actcaaacaacaggaggcacggcaaatacgcagactctgggcttc
        T Q T T G G T A N T Q T L G F

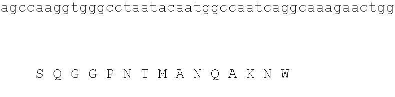

ctgccaggaccctgttaccgccaacaacgcgtctcaacgacaacc
        L P G P C Y R Q Q R V S T T T
gggcaaaacaacaatagcaactttgcctggactgctgggaccaaa
        G Q N N N S N F A W T A G T K
taccatctgaatggaagaaattcattggctaatcctggcatcgct
        Y H L N G R N S L A N P G I A

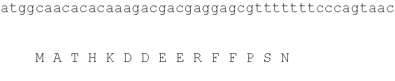

gggatcctgatttttggcaaacaaaatgctgccagagacaatgcg
        G I L I F G K Q N A A R D N A

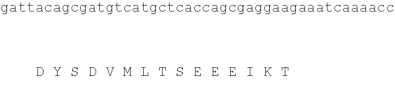

actaaccctgtggctacagaggaatacggtatcgtggcagataac
        T N P V A T E E Y G I V A D N

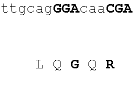

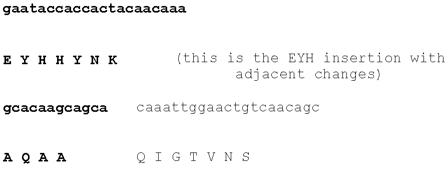

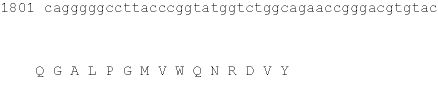



ctgcagggtcccatctgggccaagattcctcacacggacggcaac
        L Q G P I W A K I P H T D G N

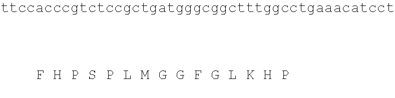

ccgcctcagatcctgatcaagaacacgcctgtacctgcggatcct
        P P Q I L I K N T P V P A D P
ccgaccaccttcaaccagtcaaagctgaactctttcatcacgcaa
        P T T F N Q S K L N S F I T Q
tacagcaccggacaggtcagcgtggaaattgaatgggagctgcag
        Y S T G Q V S V E I E W E L Q
aaggaaaacagcaagcgctggaaccccgagatccagtacacctcc
        K E N S K R W N P E I Q Y T S
aactactacaaatctacaagtgtggactttgctgttaatacagaa
        N Y Y K S T S V D F A V N T E
ggcgtgtactctgaaccccgccccattggcacccgttacctcacc
        G V Y S E P R P I G T R Y L T

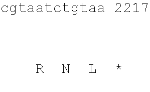

SEQ ID NOs: 7 and 8
SEQ ID NO: 7: AAV8 capsid coding (nucleotide) sequence dual modifications: (1)
Tyr447 and Tyr 733 mutated to Phe residues (bolded and in capital letters) (2)
with EYH modification (bolded and in capital letters) showing 7 additional amino
acids (EYHHYNK) being inserted after position 590, as well as adjacent changes
SEQ ID NO: 8: AAV8 capsid amino acid sequence dual modifications: (1) Tyr447 and
Tyr 733 mutated to Phe residues (bolded and in capital letters) (2) with EYH
modification (bolded and in capital letters) showing 7 additional amino acids
(EYHHYNK) being inserted after position 590, as well as adjacent changes

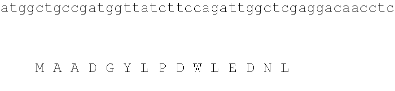

tctgagggcattcgcgagtggtgggcgctgaaacctggagccccg
        S E G I R E W W A L K P G A P
aagcccaaagccaaccagcaaaagcaggacgacggccggggtctg
        K P K A N Q Q K Q D D G R G L
gtgcttcctggctacaagtacctcggacccttcaacggactcgac
        V L P G Y K Y L G P F N G L D
aagggggagcccgtcaacgcggcggacgcagcggccctcgagcac
        K G E P V N A A D A A A L E H
gacaaggcctacgaccagcagctgcaggcgggtgacaatccgtac
        D K A Y D Q Q L Q A G D N P Y
ctgcggtataaccacgccgacgccgagtttcaggagcgtctgcaa
        L R Y N H A D A E F Q E R L Q
gaagatacgtcttttgggggcaacctcgggcgagcagtcttccag
        E D T S F G G N L G R A V F Q
gccaagaagcgggttctcgaacctctcggtctggttgaggaaggc
        A K K R V L E P L G L V E E G
gctaagacggctcctggaaagaagagaccggtagagccatcaccc
        A K T A P G K K R P V E P S P
cagcgttctccagactcctctacgggcatcggcaagaaaggccaa
        Q R S P D S S T G I G K K G Q
cagcccgccagaaaaagactcaattttggtcagactggcgactca
        Q P A R K R L N F G Q T G D S
gagtcagttccagaccctcaacctctcggagaacctccagcagcg
        E S V P D P Q P L G E P P A A

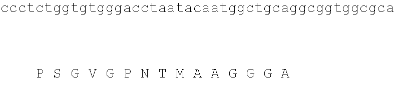

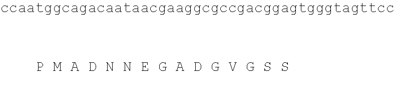


tcgggaaattggcattgcgattccacatggctgggcgacagagtc
        S G N W H C D S T W L G D R V
atcaccaccagcacccgaacctgggccctgcccacctacaacaac
        I T T S T R T W A L P T Y N N
cacctctacaagcaaatctccaacgggacatcgggaggagccacc
        H L Y K Q I S N G T S G G A T
aacgacaacacctacttcggctacagcaccccctgggggtatttt
        N D N T Y F G Y S T P W G Y F
gactttaacagattccactgccacttttcaccacgtgactggcag
        D F N R F H C H F S P R D W Q
cgactcatcaacaacaactggggattccggcccaagagactcagc
        R L I N N N W G F R P K R L S
ttcaagctcttcaacatccaggtcaaggaggtcacgcagaatgaa
        F K L F N I Q V K E V T Q N E
ggcaccaagaccatcgccaataacctcaccagcaccatccaggtg
        G T K T I A N N L T S T I Q V
tttacggactcggagtaccagctgccgtacgttctcggctctgcc
        F T D S E Y Q L P Y V L G S A

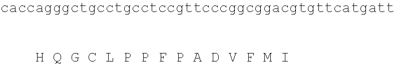

ccccagtacggctacctaacactcaacaacggtagtcaggccgtg
        P Q Y G Y L T L N N G S Q A V

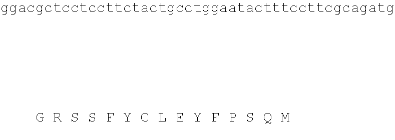

ctgagaaccggcaacaacttccagtttacttacaccttcgaggac
        L R T G N N F Q F T Y T F E D
gtgcctttccacagcagctacgcccacagccagagcttggaccgg
        V P F H S S Y A H S Q S L D R

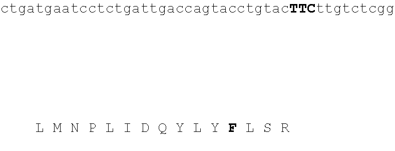

actcaaacaacaggaggcacggcaaatacgcagactctgggcttc
        T Q T T G G T A N T Q T L G F

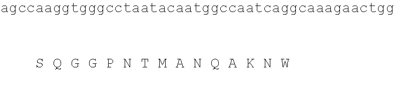

ctgccaggaccctgttaccgccaacaacgcgtctcaacgacaacc
        L P G P C Y R Q Q R V S T T T
gggcaaaacaacaatagcaactttgcctggactgctgggaccaaa
        G Q N N N S N F A W T A G T K
taccatctgaatggaagaaattcattggctaatcctggcatcgct
        Y H L N G R N S L A N P G I A

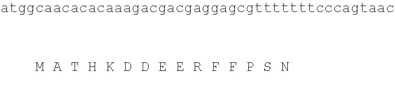

gggatcctgatttttggcaaacaaaatgctgccagagacaatgcg
        G I L I F G K Q N A A R D N A

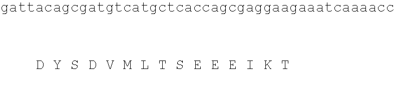

actaaccctgtggctacagaggaatacggtatcgtggcagataac
        T N P V A T E E Y G I V A D N

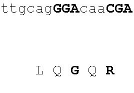

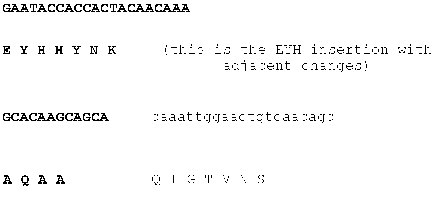

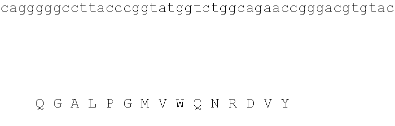



ctgcagggtcccatctgggccaagattcctcacacggacggcaac
        L Q G P I W A K I P H T D G N

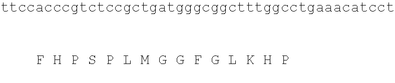

ccgcctcagatcctgatcaagaacacgcctgtacctgcggatcct
        P P Q I L I K N T P V P A D P
ccgaccaccttcaaccagtcaaagctgaactctttcatcacgcaa
        P T T F N Q S K L N S F I T Q
tacagcaccggacaggtcagcgtggaaattgaatgggagctgcag
        Y S T G Q V S V E I E W E L Q
aaggaaaacagcaagcgctggaaccccgagatccagtacacctcc
        K E N S K R W N P E I Q Y T S
aactactacaaatctacaagtgtggactttgctgttaatacagaa
        N Y Y K S T S V D F A V N T E

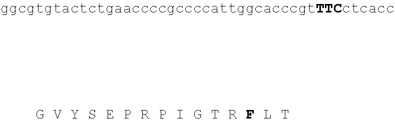

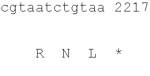


SEQ ID NO: 9
Remains of pGL3 polylinker:
ggtaccgagc tcttacgcgt gctagccc ctgcag
(KpnI NheI PstI)
SEQ ID NO: 10
LT (left-terminal) ITR (Inverted Terminal Repeat):
ttggccactc cctctctgcg cgctcgctcg ctcactgagg ccgggcgacc aaaggtcgcc
cgacgcccgg gctttgcccg ggcggcctca gtgagcgagc gagcgcgcag agagggagtg
gccaactcca tcactagggg ttcct
SEQ ID NO: 11
Sequence between ITRs:
gcggccgc atgcat agatct acgcgt ctcgag
(NotI NsiI BglII MluI XhoI)
SEQ ID NO: 12
RT (right-terminal) ITR (Inverted Terminal Repeat) plus remains of pGL3:
aggaac ccctagtgat ggagttggcc
actccctctc tgcgcgctcg ctcgctcact gaggccgggc gaccaaaggt cgcccgacgc
ccgggctttg cccgggcggc ctcagtgagc gagcgagcgc gcagagaggg agtggccaa
ctgcag gaattc ggatccg tcgaccgatg cccttgagag ccttcaaccc
(PstI EcoRI BamHI - remains of pGL3)
agtcagctcc ttccggtggg cgcggggcat gactatcgtc gccgcactta tgactgtctt
ctttatcatg caactcgtag gacaggtgcc ggcagcgctc ttccgcttcc tcgctcactg
actcgctgcg ctcggtcgtt cggctgcggc gagcggtatc agctcactca aaggcggtaa
tacggttatc cacagaatca ggggataacg caggaaagaa catgtgagca aaaggccagc
aaaaggccag gaaccgtaaa aaggccgcgt tgctggcgtt tttccatagg ctccgccccc
ctgacgagca tcacaaaaat cgacgctcaa gtcagaggtg gcgaaacccg acaggactat
aaagatacca ggcgtttccc cctggaagct ccctcgtgcg ctctcctgtt ccgaccctgc
cgcttaccgg atacctgtcc gcctttctcc cttcgggaag cgtggcgctt tctcaatgct
cacgctgtag gtatctcagt tcggtgtagg tcgttcgctc caagctgggc tgtgtgcacg
aaccccccgt tcagcccgac cgctgcgcct tatccggtaa ctatcgtctt gagtccaacc
cggtaagaca cgacttatcg ccactggcag cagccactgg taacaggatt agcagagcga
ggtatgtagg cggtgctaca gagttcttga agtggtggcc taactacggc tacactagaa
ggacagtatt tggtatctgc gctctgctga agccagttac cttcggaaaa agagttggta
gctcttgatc cggcaaacaa accaccgctg gtagcggtgg tttttttgtt tgcaagcagc
agattacgcg cagaaaaaaa ggatctcaag aagatccttt gatcttttct acggggtctg
acgctcagtg gaacgaaaac tcacgttaag ggattttggt catgagatta tcaaaaagga
tcttcaccta gatcctttta aattaaaaat gaagttttaa atcaatctaa agtatatatg
agtaaacttg gtctgacagt taccaatgct taatcagtga ggcacctatc tcagcgatct
gtctatttcg ttcatccata gttgcctgac tccccgtcgt gtagataact acgatacggg
agggcttacc atctggcccc agtgctgcaa tgataccgcg agacccacgc tcaccggctc
cagatttatc agcaataaac cagccagccg gaagggccga gcgcagaagt ggtcctgcaa
ctttatccgc ctccatccag tctattaatt gttgccggga agctagagta agtagttcgc
cagttaatag tttgcgcaac gttgttgcca ttgctacagg catcgtggtg tcacgctcgt
cgtttggtat ggcttcattc agctccggtt cccaacgatc aaggcgagtt acatgatccc
ccatgttgtg caaaaaagcg gttagctcct tcggtcctcc gatcgttgtc agaagtaagt
tggccgcagt gttatcactc atggttatgg cagcactgca taattctctt actgtcatgc
catccgtaag atgcttttct gtgactggtg agtactcaac caagtcattc tgagaatagt
gtatgcggcg accgagttgc tcttgcccgg cgtcaatacg ggataatacc gcgccacata
gcagaacttt aaaagtgctc atcattggaa aacgttcttc ggggcgaaaa ctctcaagga
tcttaccgct gttgagatcc agttcgatgt aacccactcg tgcacccaac tgatcttcag
catcttttac tttcaccagc gtttctgggt gagcaaaaac aggaaggcaa aatgccgcaa
aaaagggaat aagggcgaca cggaaatgtt gaatactcat actcttcctt tttcaatatt
attgaagcat ttatcagggt tattgtctca tgagcggata catatttgaa tgtatttaga
aaaataaaca aataggggtt ccgcgcacat ttccccgaaa agtgccacct gacgcgccct
gtagcggcgc attaagcgcg gcgggtgtgg tggttacgcg cagcgtgacc gctacacttg
ccagcgccct agcgcccgct cctttcgctt tcttcccttc ctttctcgcc acgttcgccg
gctttccccg tcaagctcta aatcgggggc tccctttagg gttccgattt agtgctttac
ggcacctcga ccccaaaaaa cttgattagg gtgatggttc acgtagtggg ccatcgccct
gatagacggt ttttcgccct ttgacgttgg agtccacgtt ctttaatagt ggactcttgt
tccaaactgg aacaacactc aaccctatct cggtctattc ttttgattta taagggattt
tgccgatttc ggcctattgg ttaaaaaatg agctgattta acaaaaattt aacgcgaatt
ttaacaaaat attaacgttt acaatttccc attcgccatt caggctgcgc aactgttggg
aagggcgatc ggtgcgggcc tcttcgctat tacgccagcc caagctacca tgataagtaa
gtaatattaa ggtacgggag gtacttggag cggccgcaat aaaatatctt tattttcatt
acatctgtgt gttggttttt tgtgtgaatc gatagtacta acatacgctc tccatcaaaa
caaaacgaaa caaaacaaac tagcaaaata ggctgtcccc agtgcaagtg caggtgccag
aacatttctc tatcgata
SEQ ID NO: 13
7-amino acid peptide that can be inserted into AAV capsid:
EYHHYNK
SEQ ID NO: 14
Upstream primer to generate Lox-1 promoter:
5′-ATATGCATCTTTCTTATTTGGGGGAAG-3′
SEQ ID NO: 14
Downstream primer to generate Lox-1 promoter:
5′-ATACGCGTACTAAAAATATGTGAGCTTCTG-3′
```

What is claimed is:

1. An expression vector for treating a cardiovascular disease, the vector comprising,
a promoter comprising a nucleotide sequence about 80% to about 100% identical to the nucleotide sequence of SEQ ID NO: 1 and,
a therapeutic gene, under control of the promoter, encoding a therapeutic agent which is therapeutically effective to treat the cardiovascular disease.

2. The expression vector of claim 1, wherein the promoter is a disease specific promoter.

3. The expression vector of claim 1, wherein the promoter is a lectin-like oxidized low density lipoprotein receptor 1 (LOX-1) promoter comprising the nucleotide sequence of SEQ ID NO: 1.

4. The expression vector of claim 1, wherein the therapeutic agent is a protein, a microRNA or an siRNA.

5. The expression vector of claim 1, wherein the therapeutic agent is Interleukin 10 (IL-10).

6. The expression vector of claim 1, wherein the therapeutic agent is Interleukin 10 encoded by a gene comprising the nucleotide sequence of SEQ ID NO: 2.

7. The expression vector of claim 1, wherein the expression vector is an adeno-associated virus (AAV) vector.

8. The expression vector of claim 1, wherein the AAV vector comprises an AAV8 capsid gene.

9. The expression vector of claim 8, wherein the AAV8 capsid gene comprises a nucleotide sequence selected from the group consisting of: (i) SEQ ID NO: 3; (ii) SEQ ID NO: 5; and (iii) SEQ ID NO: 7.

10. The expression vector of claim 1, wherein the disease is atherosclerosis.

11. An expression vector for treating a cardiovascular disease, the vector comprising,
the promoter of the lectin-like oxidized low density lipoprotein receptor 1 (LOX-1) gene and,
a therapeutic gene, under control of the promoter, encoding a therapeutic agent which is therapeutically effective to treat the cardiovascular disease.

12. The expression vector of claim 11, wherein the promoter comprising the nucleotide sequence of SEQ ID NO: 1.

13. The expression vector of claim 11, wherein the therapeutic agent is a protein, a microRNA or an siRNA.

14. The expression vector of claim 11, wherein the therapeutic agent is Interleukin 10 (IL-10).

15. The expression vector of claim 11, wherein the expression vector is an adeno-associated virus (AAV) vector.

16. The expression vector of claim 11, wherein the disease is atherosclerosis.

17. A method of treating a cardiovascular disease comprising the step of administering a therapeutically effective amount of an expression vector comprising,
a promoter comprising a nucleotide sequence about 80% to about 100% identical to the nucleotide sequence of SEQ ID NO: 1 and,
a therapeutic gene, under control of the promoter, encoding a therapeutic agent which is therapeutically effective to treat the cardiovascular disease.

18. The method of claim 17, wherein the promoter is a disease specific promoter.

19. The method of claim 17, wherein the promoter is a lectin-like oxidized low density lipoprotein receptor 1 (LOX-1) promoter comprising the nucleotide sequence of SEQ ID NO: 1.

20. The method of claim 17, wherein the therapeutic agent is a protein, a microRNA or an siRNA.

21. The method of claim 17, wherein the therapeutic agent is Interleukin 10 (IL-10).

22. The method of claim 17, wherein the expression vector is an adeno-associated virus (AAV) vector.

23. The method of claim 17, wherein the disease is atherosclerosis.


SEQUENCE LISTING (15 sequences)

SEQ ID NO: 1 - DNA, 2420 bp, homo sapien
atgaggcccacctacattatgcagcgaaatctactttcctctgctgattaatatgttaatctcatttaaaagcatcctgtctgggaaccccatgacccagtcaagaagacacagaaggccagcacggtggctcacggcttgtaatcccagcactgtgggaggctgaggcaggcggatcacttgaggacaggagttcaagaccagcctggccaacatggtgaaaccctgtctctactgaaaatacaaaacaattagccgggaggggtggcgggcacctgtaatcccagctactcaggaggctgaggcaggagaattgcttgaacccaggaggcggaggttgcagtgagccgagatcatgccattgcaacccaccctgggtaacaagggcgaaactccatctcaaaaacaaacaaacaaacaaacaaacaaacaaaacacatgaaattcaccatcacaggagttattgcttggtgggtatagagtttcagttggggaagatgaaaagttctggagatggatggtggtgatggctgcacaacaatgtaaatgtacttaatgccactgaacagtacgcctaaaaatggttgaaatggtaaacactttatgttctataatattcatggcatccaaaataactgagtcactgtagaaatgataatgtgcccttttcagtgtgatatcgtttcagtattttttaattctttttttttttttttttaaagggagtctcgctctgttgcccaggctggagtacagtggcacaatctcagctcactgaaaatgaaaagcattttcattttccacctcccaaatagctgggattataggcttgcaaccaggcccagctaacttttgtatttttagtggtgacagggtttcaacaggttggccaggctggtctcgaactcctgacctcagtgatccacccccccctcagcttcccaaagtgctgagattacaggcatgagccactgcacctggccaactttttcaatttgttctcaaatactctgcacataataaaaggaatataggaaacagggaggaggagggaaatggaatcctaattaggctgtcaatattggtaatttttcctaatagtttcctatatgttttatttatgtatttatttttccattcttcctttgtgtattcttttctttgcacttttttttcctggagtaatttaaagtaaatgccagatatcatatcattttgcttataaatacttccttcagaagacatctctaatagatatatatctggttaggaggttgtcaagggaaaatagaaaagtattctatcagtaaatacatatagacatacttatgggtctctcatgtaagcgtctttgtgacatgagtcaatcatggattttactgaaatacattatttgatgggtaattcaactgtgctgaatattagtactatactaataattattatttattggcttcttattatgctccaagtacaaagcttgtcatttttaaccagcgtgtcatttgaattcctatagcaacgtgacatttttcacaaacctagtgtaaaaaagcttattatttttacccagcatgtcatttgaattcctataacaatgttatcaggtagacattatccctaatttccagacatgtcatttgaattcctataacaatgttatcaggtagacattatccctaatttccagacaaagacatagattcaaagtattaagatttcccaagattgtagactaaaggaaagtactgaaactagctctttctgcattcaaagtgctctctgctgctacgttacactggcactgcatgaactagtagccccagataaattctaaaatcacccaggacacaccatcgctggaaatgggagccatatgtgattcaccccaaaattcttagatccattgccagataaaacagtgataatcaaaagagaatatatctgattcataaaagacaccatatagaaacagtctgctaatgaaattagcaaacaaacattggaatcaaatattttggcccataattctggcacattttttacaaatgtagtgtgacttactctctttgaatttcagtttctgtctctgaagagtgggtacaatatctcctctgatgctcatgaaaaatagttttccctttcataaattacttagcgaaatatcctgaaacaccttcagaatcaccactttctccacctgcaatacacataactcaagaatttgcgtcagcgaacttcccaatatgaagcaaagcctctccttcctcctaccaatgattgagccattcttctattagataacagtagctatttaaatacttctgcagaagctcacatatttttagtttgttgaagttcgtgactgcttcactctctcattcttagcttgaatttggaaatgacttttgatgacctaa

SEQ ID NO: 2 - DNA, 460 bp, homo sapien
aaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaag

SEQ ID NO: 3 - DNA, 2217 bp, artificial sequence
  AAV8 capsid coding (nucleotide) sequence with Tyr447 and Tyr 733 mutated to Phe residues
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtacttcttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgtttcctcacccgtaatctgtaa

SEQ ID NO: 4 - protein, 738 aa, artificial sequence
  AAV8 capsid amino acid sequence with Tyr447 and Tyr 733 mutated to Phe residues
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYFLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRFLTRNL

SEQ ID NO: 5 - DNA, 2241 bp, artificial sequence
  AAV8 capsid coding (nucleotide) sequence with EYH modification showing 7 additional amino acids (EYHHYNK) being inserted after position 590 as well as adjacent changes
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagggacaacgagaataccaccactacaacaaagcacaagcagcacaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 6 - protein, 743 aa, artificial sequence
  AAV8 capsid amino acid sequence with EYH modification showing 7 additional amino acids (EYHHYNK) being inserted after position 590, as well as adjacent changes
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQGQREYHHYNKAQAAQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLT

SEQ ID NO: 7 - DNA, 2241 bp, artificial sequence
  AAV8 capsid coding (nucleotide) sequence dual modifications (1) Tyr447 and Tyr 733 mutated to Phe residues (2) with EYH modification showing 7 additional amino acids (EYHHYNK) being inserted after position 590, as well as adjacent changes
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtacttcttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagggacaacgagaataccaccactacaacaaagcacaagcagcacaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgtttcctcacccgtaatctgtaa

SEQ ID NO: 8 - protein, 743 aa, artificial sequence
  AAV8 capsid amino acid sequence dual modifications (1) Tyr447 and Tyr 733 mutated to Phe residues (2) with EYH modification showing seven additional amino acids (EYHHYNK) being inserted after position 590, as well as adjacent changes
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYFLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQGQREYHHYNKAQAAQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRFLT

SEQ ID NO: 9 - DNA, 34 bp, artificial sequence
  Remains of pGL3 polylinker
ggtaccgagctcttacgcgtgctagcccctgcag

SEQ ID NO: 10 - DNA, 145 bp, artificial sequence
  LT ITR (Inverted Terminal Repeat)
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 11 - DNA, 32 bp, artificial sequence
  Sequence between ITRs
gcggccgcatgcatagatctacgcgtctcgag

SEQ ID NO: 12 - DNA, 2972 bp, artificial sequence
  RT ITR (Inverted Terminal Repeat) plus remains of pGL3
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactgcaggaattcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 13 - protein, 7 aa, artificial sequence
  7-amino acid peptide that can be inserted into AAV capsid sequence
EYHHYNK

SEQ ID NO: 14 - DNA, 27 bp, artificial sequence
  Upstream primer to generate Lox-1 promoter
atatgcatctttcttatttgggggaag

SEQ ID NO: 15 - DNA, 30 bp, artificial sequence
  Downstream primer to generate Lox-1 promoter
atacgcgtactaaaaatatgtgagcttctg